(19) United States
(12) Patent Application Publication
(10) Pub. No.: US 2025/0283961 A1
(43) Pub. Date: Sep. 11, 2025

(54) Computer-Implemented Method for Supporting and/or Assisting a User by Means of an Assistance System When Executing a Measurement Program During Magnetic Resonance Data Acquisition

(71) Applicant: Siemens Healthineers AG, Forchheim (DE)

(72) Inventors: Silvia Bettina Arroyo Camejo, Fuerth (DE); Dirk Franger, Erlangen (DE)

(73) Assignee: Siemens Healthineers AG, Forchheim (DE)

(21) Appl. No.: 19/073,247

(22) Filed: Mar. 7, 2025

(30) Foreign Application Priority Data
Mar. 8, 2024 (EP) 24162381.8

(51) Int. Cl. G01R 33/44; G16H 40/60

(52) U.S. Cl. CPC G01R 33/44; G16H 40/60

(57) ABSTRACT

Techniques are provided for supporting and/or assisting a user by means of an assistance system when executing a measurement program during magnetic resonance data acquisition.

### CROSS-REFERENCE TO RELATED APPLICATIONS

The present application claims priority to and the benefit of European patent application no. EP 24162381.8, filed on Mar. 8, 2024, the contents of which are incorporated herein by reference in their entirety.

### TECHNICAL FIELD

The present disclosure concerns a computer-implemented method for supporting and/or assisting a user by means of an assistance system when executing a measurement program during magnetic resonance data acquisition. Moreover, the present disclosure also concerns an assistance system designed to execute the method for supporting and/or assisting a user when executing a measurement program during magnetic resonance data acquisition. The disclosure is also based on a magnetic resonance device with an assistance system. Moreover, the present disclosure also concerns a computer program product, comprising a program and which may be loaded directly into a memory of a programmable control unit, with program means for executing a method for supporting and/or assisting a user by means of an assistance system when executing a measurement program for magnetic resonance data acquisition, when the program is executed in the control unit.

### BACKGROUND

Diagnostic imaging devices, such as magnetic resonance devices, provide extensive structural and functional information on a patient allowing a doctor to make a diagnosis. This information exists in the form of image data, e.g. magnetic resonance image data. The accuracy of a medical diagnosis is highly dependent upon the quality of the recorded medical image data. At the same time, the quality of the medical image data is dependent upon various factors. One factor may be a hardware configuration of the magnetic resonance device. At the same time, BO magnetic field inhomogeneity and/or gradient field strength and/or the quality and robustness of accessories such as local coils may have a strong influence on both the average image quality and also the variability of the image quality of the acquired magnetic resonance image data.

Another factor affecting the image quality is the patient. For example, patient motion may have a considerable effect on the image quality and, for example, lead to image artifacts. When imaging the abdomen, during which the patient is to perform defined respiratory movements, errors, such as incorrect breathing, may also result in impaired image quality. Another example is imaging of the heart, during which an ECG signal triggers and/or initiates the imaging. In patients with a cardiac arrhythmia, however, this may lead to incorrect triggering of image data acquisition.

An additional aspect influencing the image quality is the medical operating personnel performing or overseeing the magnetic resonance examination on a patient. For optimum operation of a magnetic resonance device, e.g. for optimum setting and/or adaptation of operating parameters and/or measurement parameters, medical operating personnel must have many years of experience. Less experienced medical operating personnel, on the other hand, often use the standard settings without utilizing the full potential of the magnetic resonance device.

To support medical operating personnel, software components are known that are able to partially compensate for hardware-related defects or artifacts in the image data. Examples of these are distortion correction algorithms in the image reconstruction, which compensate for image artifacts resulting from BO magnetic field inhomogeneity. Furthermore, measurement programs and/or measurement sequences are known which are particularly robust against patient motion during magnetic resonance data acquisition or allow freedom of breathing during magnetic resonance data acquisition in investigations of the abdomen and/or of the heart. However, an optimized adaptation of measurement sequences for the patient is not available.

### SUMMARY

The object of the present disclosure is to provide simple and automated support for a user when executing a measurement program. The object is achieved by the features of the embodiments as discussed herein, including the claims.

The disclosure is based on a computer-implemented method for supporting and/or assisting a user by means of an assistance system when executing a measurement program during magnetic resonance data acquisition, comprising the following method steps:
Selecting a measurement program for acquisition of magnetic resonance data with a defined diagnostic question;
Executing the selected measurement program, the execution of the selected measurement program comprising acquisition of magnetic resonance data and/or reconstruction of image data from the acquired magnetic resonance data, and provision of the magnetic resonance data and/or the reconstructed image data;
Acquiring further measurement information, the further measurement information being acquired before execution of the selected measurement program or during execution of the selected measurement program, and provision of the further measurement information;
Determining at least one item of evaluation information by means of at least one analysis module of the assistance system as a function of the provided magnetic resonance data and/or the provided reconstructed image data and/or the provided further measurement information, and provision of the at least one item of evaluation information;
Generating an item of assistance information, the assistance information being generated as a function of the at least one item of evaluation information by means of the at least one analysis module, and provision of the assistance information; and
Outputting the assistance information via a user interface to a user.

For the magnetic resonance data acquisition a magnetic resonance examination is performed on a patient, e.g. on an area of the patient to be examined, by means of a magnetic resonance device. For a magnetic resonance examination, during which a defined clinical and/or diagnostic question is to be clarified, a measurement program is first selected based on the defined clinical and/or diagnostic question. The defined clinical and/or diagnostic question may, for example, be dependent upon a region of the patient's body to be examined and/or an illness the patient has.

Such a measurement program may comprise multiple measurement steps. The individual measurement steps may each comprise at least one magnetic resonance sequence. With multiple magnetic resonance sequences these may be executed and/or performed one after another in a defined and/or determined order. The individual magnetic resonance sequences may e.g. comprise a time sequence of high-frequency pulses. For example, a magnetic resonance sequence may comprise a T1-weighted sequence or a T2-weighted sequence or a spin echo sequence, etc. The individual magnetic resonance sequences differ with regard to their sequence parameters.

The selection of a measurement program is made by a user. At the same time, the user may also access preset measurement programs, which determine the order of measurements of the individual magnetic resonance sequences for a clinical or diagnostic question. Furthermore, an experienced user may also independently compile and/or select a measurement program from individual magnetic resonance sequences.

During execution of the measurement program the individual measurement steps, e.g. the individual measurement sequences, are executed and magnetic resonance data is acquired. For this purpose the patient, e.g. the area of the patient to be examined, is positioned within a patient receiving area of a magnetic resonance device. For example, the area of the patient to be examined is located in an isocenter of the magnetic resonance device.

During execution of the measurement program image data is also reconstructed from the acquired magnetic resonance data. At the same time, a reconstruction of the image data may take place by means of the measurement program or also by means of a reconstruction unit. The provided magnetic resonance data may e.g. comprise magnetic resonance raw data and/or k-space data.

Apart from the magnetic resonance data, further measurement information (or simply referred to herein as measurement information) may also be acquired. This further measurement information may comprise patient data, already known and/or acquired before the magnetic resonance examination. For example, this patient data may already have been acquired during patient registration, such as patient weight and/or patient height and/or patient age. Furthermore, such patient data may be known from preliminary examinations of the patient, such as a patient's cardiac arrhythmia. Furthermore, the further measurement information may only be acquired during execution of the measurement program, such as the patient's physiological data which is being continually acquired during execution of the measurement program. Such physiological data may, for example, comprise the patient's ECG data or also respiration data. Furthermore, the further measurement information, which is continuously acquired during execution of the measurement program, may comprise information with regard to patient motion.

The assistance system comprises at least one analysis module. In an embodiment, the assistance system comprises multiple different analysis modules. The individual analysis modules evaluate and/or analyze for a defined question the provided magnetic resonance data and/or the provided image data and/or the provided further measurement information. The defined question may comprise a question with regard to quality, e.g. image quality, in the provided magnetic resonance data and/or image data. Furthermore, the defined question may comprise a clinical and/or diagnostic question. Furthermore, the defined question may also comprise a technical question. Furthermore, the defined question may also comprise a general examination-related question.

To answer the respective defined question, the individual analysis modules determine evaluation information as a function of the provided magnetic resonance data and/or the provided image data and/or the provided further measurement information. To determine the evaluation information, the individual analysis modules have an evaluation algorithm. The evaluation information may, for example, comprise a categorization and/or classification of the determined answer as “Good” or “Poor”. Furthermore, the evaluation information may also comprise a multiclass classification, in which the evaluation information is present in the format (1,0,0, . . . , 0), (0,1,0, . . . , 0), (0,0,1, . . . , 0), . . . (0,0,0, . . . , 1). Furthermore, the evaluation information may also comprise a multiclass-multilabel classification, in which the evaluation information is present in the format (1,0,0, . . . , 0), (1,1,0, . . . , 0) (1,1,1, . . . , 0), . . . (1,1,1, . . . , 1). In all cases, the evaluation information may also take the value “−1” if, for example, no analysis and/or determination of an item of evaluation information was possible and/or an analysis by means of the at least one analysis module was discontinued. Furthermore, the evaluation information may comprise further categorization and/or classification deemed useful by the person skilled in the art.

Apart from the analysis modules, the assistance system may also have further modules and units. In an embodiment, the assistance system has a transaction manager, the transaction manager being designed to provide the provided magnetic resonance data and/or the provided image data and the provided further measurement information to the at least one analysis module or the multiple analysis modules. Furthermore, the transaction manager may regulate and/or control a data exchange between individual analysis modules or between analysis modules and further modules. For example, the transaction manager may also provide further modules with the evaluation information from analysis modules. For this purpose, the individual analysis modules and/or the further modules may each have a corresponding interface, which communicates with the transaction manager. For example, the transaction manager may also control an execution of the individual analysis modules and/or an order of execution of individual analysis modules.

Furthermore, the assistance system may also comprise a configuration user interface, which may be designed to provide a user during configuration and/or selection of the measurement program with a selection of analysis modules, the user being able from this selection to select an analysis module or also multiple analysis modules for the provision of assistance information. In an embodiment, the user may, by means of the configuration user interface, configure support, e.g. a selection of at least one analysis module and/or at least one further module, at a determined and/or defined position in the measurement program. At the same time, the user may with some analysis modules also have the opportunity, by means of the configuration user interface, to select a data type and/or a data format of input data for the selected module. A further possibility by means of the configuration user interface is that the user may also configure individual analysis modules, e.g. adapt these to at least one measurement step. The user may also configure and/or select a linking of multiple analysis modules.

Moreover, the configuration user interface may also be designed to specify a data format of input data and/or output data of analysis modules. Furthermore, the configuration user interface may also be designed to automatically specify a selection of analysis modules, the user being able from this selection to select at least one analysis module for supporting the measurement program.

With the aid of the evaluation information, assistance information is also generated. The assistance information is generated by the analysis module based on the at least one item of evaluation information. The assistance information is intended for output to a user. The assistance information may indicate to, or inform, the user if, during the magnetic resonance examination, e.g. the execution of the measurement program, problems and/or critical situations and/or abnormalities have arisen that adversely affect a magnetic resonance data acquisition and/or the execution of the measurement program. Furthermore, the assistance information may also inform the user that critical situations and/or abnormalities have been detected and that these may lead to a potential problem with the execution of the measurement program and/or the magnetic resonance data acquisition. Furthermore, the assistance information may also be designed to designate a type of problem and/or critical situation and/or abnormality. For example, the assistance information may indicate to the user a poor image quality in the acquired magnetic resonance data and/or the provided image data and also indicate to the user a possible, but undesired patient motion during the execution of the measurement program, the patient motion being the possible cause of the poor image quality.

In an embodiment, the at least one analysis module is designed to execute the analysis of the provided magnetic resonance data and/or the provided reconstructed image data and/or the provided further measurement information continuously throughout the execution of at least one measurement step so that if a spontaneous problem and/or a critical situation occurs the user may be immediately informed.

The output of the assistance information may e.g. take place via a user interface of the magnetic resonance device. The assistance information may e.g. be output via a visual display unit, for example a monitor and/or a touch display. The user may be e.g. a member of the medical operating personnel overseeing the magnetic resonance examination. The member of the medical operating personnel may, for example, be a doctor, such as a radiologist, or a medical radiology technologist.

The disclosure has the advantage that a user during a magnetic resonance examination, e.g. execution of the measurement program, may be immediately informed of problems and/or critical situations and/or abnormalities during the magnetic resonance data acquisition. Consequently, the user receives immediate feedback and based on the information, e.g. the assistance information, and may decide whether they wish to take measures for correcting the error or problem, for example by repeating individual measurement steps of the measurement program or changing the recording parameters. For example, the user may also change the time for which the patient holds their breath if they are having problems holding their breath for a long time. Moreover, the user may also select an alternative measurement sequence for the measurement step. Furthermore, the user may also decide that despite the quality of the image data being poor, it is nevertheless adequate for assessing the clinical or diagnostic question. Furthermore, based on the information, e.g. the assistance information, the user may decide if they wish to take measures for a more detailed analysis of the abnormalities detected in the image data, for example further measurement steps, focusing on an image area with the abnormality.

By immediately informing the user, e.g. by the output of the assistance information while the measurement program is running, effort, e.g. examination effort, may be reduced for the patient and the user. If, for example, the acquired magnetic resonance data and/or image data reconstructed from the magnetic resonance data is unsuitable for a diagnostic assessment, the measurement step or multiple measurement steps may be repeated and/or executed with changes. A repeat only after days or weeks, once the magnetic resonance data and/or image data has been analyzed and a repeat appointment found for the patient, may thereby be avoided. For example, only the relevant, e.g. only the defective and/or flawed measurement steps, need be repeated and not the entire measurement program.

Furthermore, it also may be provided that the assistance system, apart from the at least one analysis module, comprises at least one recommendation module, that the recommendation module based on the provided evaluation information and/or the provided assistance information determines a recommendation and/or a suggestion and provides this to the user. This recommendation and/or the suggestion may, for example, comprise a further action for the measurement program. This also enables inexperienced users, if abnormalities occur in the acquired magnetic resonance data and/or the further measurement information, to successfully complete the measurement program and to prepare for a subsequent diagnosis.

Alternatively or in addition, it may be provided that the at least one item of assistance information is output to a user via a user interface for controlling and/or monitoring the measurement program. The user interface for controlling and/or monitoring the measurement program may e.g. display the individual measurement steps of the measurement program. At the same time, for the individual measurement steps, the analysis modules activated and/or selected for the measurement step may also be displayed. In an embodiment, the assistance information for the respective measurement step is displayed in the user interface. In an embodiment, the assistance information is displayed and/or output at a defined position at the user interface. In this way simple and rapid transmission of the assistance information to the user may take place. In an embodiment, the user may concentrate on a single user interface and receive all information available for the measurement program including the assistance information.

Alternatively or in addition, it may be provided that the assistance system also comprises its own user interface for output of information, e.g. the assistance information. Such a user interface may, for example, comprise an assistance user interface, which apart from the assistance information may also inform the user which analysis module is currently being executed and for which measurement step this was selected.

Alternatively or in addition, it may be provided that the at least one further item of evaluation information is determined by a further analysis module as a function of the provided evaluation information of the at least one analysis module. For example, the provided evaluation information of the first analysis module may comprise the input data for the further analysis module. Furthermore, the input data for the further analysis module may comprise both the provided evaluation information and the provided magnetic resonance data and/or the provided reconstructed image data and/or the provided further measurement information. In an embodiment, the further analysis module builds on the first analysis module to clarify a clinical question and/or a technical question and/or a patient-related question. The assistance information may e.g. be generated by the further analysis module. In this way advantageous support of a user even for complex questions and/or complex situations, e.g. complex measurement situations, may be provided.

Alternatively or in addition, it may be provided that for generating the assistance information at least two items of evaluation information are received from at least two analysis modules. For instance, each of the at least two analysis modules may determine an item of evaluation information independently of the further analysis modules. The assistance information is then generated from all the evaluation information available. The assistance information may, for example, be generated by means of artificial intelligence (AI). At the same time, it may be that for generation of the assistance information the AI weights the individual items of evaluation information differently. Weighting of the individual evaluation information may be dependent upon the type of measurement program, e.g. the individual measurement steps and/or the type of analysis module.

In an advantageous development of the method, it may be provided that the assistance information comprises status information of the analysis module. The status information of the analysis module may show the user what type the analysis module is, for example if a quality analysis module and/or a technical analysis module and/or a clinical analysis module has been selected for the assessment and/or analysis of the measurement step. Furthermore, the status information may show the user if an analysis by means of the analysis module is currently being carried out. Furthermore, the status information may show the user if the analysis by means of the analysis module has been successfully completed and a result is available. Furthermore, the status information may show the user if the analysis by means of the analysis module could not be carried out. In an embodiment, the status information of the analysis module is shown by corresponding symbols, which advantageously are arranged at a defined and/or specified position on the user interface. This allows the user to be provided with a rapid overview of the analysis module.

Alternatively or in addition, it may be provided that the assistance information comprises problem information and/or abnormality information. The problem information comprises information indicating to the user a problem and/or a critical state and/or an abnormality during the execution of the measurement program. For example, a problem detected by the analysis module and/or a critical state detected by the analysis module may comprise poor image quality and/or patient motion. Furthermore, a problem detected by the analysis module and/or a critical state detected by the analysis module may be incorrect breathing by the patient, for example during cardiac imaging. Furthermore, the problem information may comprise information that no problem and/or no critical state has occurred during the execution of the measurement program and/or been detected by the analysis module. The abnormality information may e.g. comprise information describing an abnormality of the patient, and hence a state deviating from a norm. Such abnormality information does not necessarily represent a quality problem in the acquired data. Rather, the abnormality information may also comprise neutral information indicating, for example, an anomaly and an abnormality during the execution of at least one measurement step of the selected measurement program. Furthermore, the abnormality information may also comprise information that no abnormality was detected in the analyzed data. If an abnormality is present an adaptation of the further measurement program may also be advisable. For example, if an anomaly is detected, additional image data may be provided in a further measurement step to enable a radiologist, for example, to subsequently make a diagnosis. At the same time, it is not always necessary to adapt the measurement program, e.g. the further steps of the measurement program, in the event of an abnormality. In this way, extensive information may be provided to a user on the current magnetic resonance data acquisition. For instance, the user may decide on possible alternatives for improving magnetic resonance data acquisition and/or for correcting the problem that has occurred and/or an existing abnormality based on the problem information and/or abnormality information provided.

For example, in a head examination, the assistance information may include information that a non-specific mass has been detected in the patient's brain. For further analysis or for diagnosis of the non-specific mass the user may incorporate further measurement steps that focus on detection of the area of the brain with the abnormality in the measurement program. Furthermore, provided the assistance system comprises a recommendation module, the user may be provided with a corresponding suggestion for adapting the measurement program, e.g. by adding further measurement steps, that focus on detection of the area of the brain with the abnormality. A further example would be if the analysis of the image data reveals that no abnormality is present. If, for example, individual measurement steps comprise a contrast medium measurement, to better highlight abnormalities in the tissue being examined, such measurement steps may be omitted by the user. Furthermore, provided the assistance system comprises a recommendation module, the user may be provided with a corresponding suggestion for adapting the measurement program, e.g. by removing contrast medium-supported steps.

Alternatively or in addition, it may be provided that the output of the problem information and/or abnormality information to a user interface comprises an active retrieval by the user. In an embodiment, the problem information and/or abnormality information is provided to a user at the user interface, e.g. at an operator interface, via the assistance information. For example, the active retrieval by the user may comprise actively clicking a button on the user interface, the active clicking causing and/or initiating the display of the problem information and/or abnormality information. In an embodiment, for this purpose the user interface, via which the problem information and/or abnormality information is retrievable, has a symbol linked to the problem information and/or abnormality information, and/or an object linked to the problem information and/or abnormality information, that may be clicked or selected by a user, for example by means of a computer mouse. This allows the information required by the user, e.g. the problem information and/or abnormality information, to be displayed. If the user has no need to view the problem information and/or abnormality information, an uncluttered user interface may be provided without excessive additional information.

In an advantageous development of the method, it may be provided that the at least one analysis module comprises at least one quality analysis module and/or at least one clinical analysis module and/or at least one technical analysis module and/or at least one general analysis module.

The at least one quality analysis module is designed to identify certain characteristics in input data, the certain characteristics relating to certain or specific data quality problems. At the same time, the at least one quality analysis module is designed to evaluate the severity of the data quality problem. If reconstructed image data is available as input data of the at least one quality analysis module, the at least one quality analysis module may be designed to identify motion artifacts and/or Gibbs artifacts, and so on, in the reconstructed image data. In an embodiment, the at least one quality analysis module is designed to check and analyze the input data continuously with regard to the particular or specific data quality problem. The presence of a data quality problem may, for example, adversely affect the image quality in the reconstructed image data such that a diagnostic evaluation of the image data is no longer possible or there is a danger of misdiagnosis due to the image quality. An example of this might be strong motion artifacts during the image data acquisition. For example, a quality analysis module may comprise a trained quality analysis module, trained to infer patient motion in the image artifacts present in the reconstructed image data.

Furthermore, the at least one quality analysis module may also monitor and analyze further measurement information, for example physiological data of the patient, during the execution of the measurement program. Such physiological data may, for example, be ECG data of the patient, acquired from the patient during the execution of the measurement program. Furthermore, such physiological data may also comprise data from the monitoring of the patient's breathing and/or further physiological data deemed useful by the person skilled in the art. The at least one quality analysis module may, for example, determine a probability of a problem and/or a critical state occurring in the image data, if no countermeasures are taken. For example, a detection algorithm, that analyzes the patient's ECG signal during the execution of the measurement program, to detect possible cardiac arrhythmias, may determine from this the probability of possible motion artifacts. A measure of the deviation of the physiological signal from an expected normal physiological signal may be included in the probability determination. If the probability exceeds a limit, this is evaluated and/or detected as a quality problem by the at least one quality analysis module, which is reflected in the evaluation information of the at least one quality analysis module.

The at least one quality analysis module may also determine the image quality in the image data. At the same time, the at least one quality analysis module may, for example, consider one of the following image quality characteristics:

Normalized root mean square (NRMS), also referred to as scatter index, is a statistical error indicator.

Peak signal-to-noise ratio (PSNR), is the ratio between the maximum possible power of a signal and the strength of the corrupting noise.

DCT (discrete cosine transform) sub-bands similarity (DSS). DSS exploits important features of human visual perception by measuring changes in structural information in sub-bands in the DCT range and weighting the quality estimates for these sub-bands.

Gradient magnitude similarity deviation (GMSD). Image gradients react sensitively to image distortions, during which different local structures in a distorted image are affected to varying degrees. The GMSD uses the pixel-by-pixel gradient magnitude similarity (GMS) combined with the standard deviation to calculate an image quality index.

Haar wavelet-based perceptual similarity index (HaarPSI) is a similarity measure for images that aims to correctly assess the perceptual similarity of two images in relation to a human observer.

Mean deviation similarity index (MDSI) uses the gradient size to measure structural distortion and colorfulness characteristics to measure color distortion in images. These two similarity maps are combined to form a gradient-chromaticity similarity map and the final quality assessment is calculated from this.

Mean structural similarity index measure (MSSIM) measures the similarity between two given images.

Multi-scale structural similarity index measure (MSSSIM) is a more advanced form of SSIM that is performed using multiple scales in a process with multilevel reduction of sampling.

Visual information fidelity (VIF) is a complete reference index for evaluating image quality based on natural scene statistics and the presentation of image information extracted from the human visual system.

Visual saliency-based index (VSI). Here the visual characteristics are used to calculate a local quality map of the distorted image. Furthermore, the visual expression is used as a weighting function when summarizing the quality factor to reflect the importance of a local region.

Deep image structure and texture similarity (DISTS) describes an image quality method that combines correlations of spatial averages (“texture similarity”) with correlations in feature maps (“structure similarity”).

Learned perceptual image patch similarity (LPIPS) is used to assess the perceptual similarity between two images. LPIPS essentially calculates the similarity between the activations of two image patches for a predefined network.

Perceptual image error metric (PieAPP) measures the perception error of a distorted image in relation to a reference and the associated data set.

Total variation (TV) identifies several slightly different concepts that relate to the structure of the range of values of a function or measure.

Blind referenceless image spatial quality evaluator (BRISQUE) is a model that uses only the image pixels to calculate characteristics. It has proven extremely efficient since no transformation is required for calculating its characteristics. It is based on the spatial NSS (Natural Scene Statistics) model of locally normalized luminance coefficients in the spatial domain, and on the model for paired products of these coefficients.

Natural image quality evaluator (NIQE) measures the gap between the NSS-based characteristics calculated from an image and the characteristics obtained from an image database and used to train the model. The characteristics are modelled as multidimensional Gaussian distributions.

The at least one clinical analysis module may be designed to analyze the input data, e.g. the reconstructed image data and/or k-space data, for abnormalities. Such abnormalities may point to an illness, for example a suspected hemorrhage and/or a suspected heart attack. With the aid of the input data of the analysis module, e.g. the reconstructed image data, a probability may be determined for the presence of an abnormality in the reconstructed image data by the at least one technical analysis module. If the determined probability exceeds a limit, this is evaluated or detected by the at least one clinical analysis module as an abnormality and/or anomaly, which is reflected in the evaluation information by the at least one clinical analysis module. At the same time, no diagnosis of the input data, e.g. the reconstructed image data, takes place by means of the clinical analysis module, it is merely an aid to a diagnosis by a doctor. In an embodiment, the analysis modules and the provided evaluation information and/or the provided assistance information are intended to provide a doctor making a diagnosis with maximum support, for example by also providing image data of a detected abnormality and/or anomaly during the diagnosis, which would not necessarily be available for the originally selected measurement program without adaptation based on the assistance information.

The at least one technical analysis module may be e.g. designed to analyze technical and/or device-related information, available to the measurement program. In an embodiment, the at least one technical analysis module is designed for analysis of coil data of local high frequency coils. The local high frequency coils are positioned for acquisition of magnetic resonance data around the area to be examined of the patient. For different body areas, there are also different local high frequency coils available, for example, a head high frequency coil for a head examination or a knee high frequency coil for a knee examination. Based on the coil data the at least one technical analysis module may, for example, detect if for the selected measurement program the high frequency coil needed for this is connected with a scanner unit of a magnetic resonance device. Furthermore, the technical analysis module may also be designed for detection and/or early detection of hardware defects. For example, the at least one technical analysis module may be designed to analyze current operating parameters of local high frequency coils and to infer from this a status, e.g. a remaining service life, of the local high frequency coils or of individual components of the local high frequency coils.

The at least one general analysis module may be designed to monitor and to analyze general processes associated with the execution of the measurement program. At the same time, the at least one general analysis module may comprise a technical detection algorithm. Such a technical detection algorithm may, for example, be designed to detect and/or analyze the triggering of an image pre-processing. Alternatively or in addition, the at least one general analysis module may also comprise a setting-dependent detection algorithm. Such a setting-dependent detection algorithm may, for example, compare settings of the selected measurement program with existing software configurations and/or software licenses and/or with scanner configurations and in the event of deviations generate a warning that for execution of the measurement program not all software-relevant and/or scanner-relevant criteria have been met and therefore restrictions and/or problems may be expected during the execution of the measurement program. Alternatively or in addition, the at least one general analysis module may also be designed for analysis of general processes of the measurement program, such as an analysis for reporting.

This embodiment of the disclosure has the advantage that for a broad spectrum of possible problems and/or a broad spectrum of critical states during the execution of the selected measurement program support, for example suggesting an adaptation of the selected measurement program based on the provided assistance information, is offered by the assistance system. This allows the user, e.g. a member of the medical operating personnel overseeing the magnetic resonance examination, to be warned of possible complications of various kinds during the execution of the measurement program. This also enables early initiation of countermeasures by the user, so that despite the presence of complications and/or problems the measurement program may be executed.

Alternatively or in addition, it may be provided that the at least one analysis module analyzes a defined clinical question and/or a defined technical question and/or a defined patient-related question as a function of the selected measurement program. In an embodiment, the at least one analysis module is coordinated with the selected measurement program. The selected measurement program is selected based on a defined clinical and/or diagnostic question. The at least one analysis module is also coordinated with the clinical and/or diagnostic question of the selected measurement program. If, for example, the selected measurement program comprises a head examination, the defined clinical question of the at least one analysis module may comprise an analysis of head images. At the same time, the head images may be monitored and/or analyzed for abnormalities and/or irregularities. Furthermore, the defined clinical question of the at least one analysis module may also comprise a time of a contrast medium administration. At the same time, the at least one analysis module may also monitor and/or analyze, if a contrast medium has been administered at the correct time, and so on. In an embodiment, the at least one analysis module comprises at least one quality analysis module and/or a clinical analysis module for analysis of the defined clinical question.

Furthermore, apart from a defined clinical question a defined technical question in connection with the selected measurement program of the at least one analysis module may be clarified. In an embodiment, the at least one analysis module for analysis of the defined technical question comprises at least one technical analysis module. For head examinations, for example, the defined technical question of the at least one technical analysis module may be “if the correct high frequency coils is being used” and/or “if the high frequency coil is correctly positioned and/or plugged in” and/or “if additional units, such as a contrast medium injector are correctly connected”, etc.

Furthermore, apart from a defined clinical question and/or a defined technical question a defined patient-relevant question in connection with the selected measurement program of the at least one analysis module may also be clarified. In an embodiment, the at least one analysis module for analysis of the defined patient-related question comprises at least one quality analysis module and/or a clinical analysis module. If, for example, it is already known that the patient has a cardiac arrhythmia then, for example, for a selected measurement program, that comprises a cardiac examination of the patient, no analysis module is selected designed to detect a cardiac arrhythmia. Instead, an analysis module is selected that already takes into account the patient's cardiac arrhythmia in the reconstructed image data during an analysis.

This allows individual support to be provided to the user when executing the selected measurement program. For instance, relevant questions for the measurement program may be monitored and/or analyzed. Furthermore, such pre-existing conditions and/or existing findings of the patient may be taken into account in the execution of the measurement program.

In an advantageous development of the method it may be provided that the at least one analysis module determines the at least one item of evaluation information by means of a rule-based algorithm and/or a machine learning-based algorithm. A rule-based algorithm is based on defined rules, in accordance with which this algorithm performs the task assigned to it. To solve the task a result may be compared with at least one threshold and from this a statement may be derived, e.g. the evaluation information.

A machine learning-based algorithm may e.g. comprise a trained machine learning method and has been trained to detect certain characteristics and/or a certain pattern in the input data to be analyzed. In general, a trained machine learning process mimics cognitive functions that humans associate with other human thoughts. In an embodiment, the training based on training data allows the machine learning method to adapt to new conditions and to detect and extrapolate patterns. Another term for “trained machine-learning method” is a “trained function” or a “trained machine-learning model”. In general, parameters of a machine learning method may be adapted through training. In an embodiment, monitored training, partly monitored training, unmonitored training, reinforcement learning, and/or active learning may be used. In addition, representation learning (or feature learning) may be used. In an embodiment, the parameters of the machine learning method may be adapted iteratively through multiple training steps. Moreover, within the training of the neural network the backpropagation-algorithm may be used. In an embodiment, a machine learning method may comprise a neural network, a support vector machine, a decision tree and/or a Bayesian network, and/or the machine learning method may be based on k-means clustering, Q-learning, genetic algorithms and/or association rules. In an embodiment, a neural network may be a deep neural network, a convolutional neural network, or a convolutional deep neural network. In addition, a neural network may be a contradictory network, a deep contradictory network, and/or a generative contradictory network.

An artificial neural network (ANN) may comprise for instance a network simulated in a computer program of artificial neurons. The artificial neural network is typically based on a networking of multiple artificial neurons. The artificial neurons are typically arranged on different layers. Normally, the artificial neural network comprises an input layer and an output layer whose neuron output is the only one visible in the artificial neural network. Layers located between the input layer and the output layer are typically referred to as hidden layers. Typically, to begin with, an architecture and/or topology of an artificial neural network is initiated and then in a training phase trained for a special task or for multiple tasks in a training phase. The training of the artificial neural network typically comprises a change to a weighting of a link between two artificial neurons of the artificial neural network. The training of the artificial neural network may also comprise development of new links between artificial neurons, deletion of existing links between artificial neurons, adaptation of thresholds of the artificial neurons and/or addition or deletion of artificial neurons. The artificial neural network has e.g. already been suitably trained in advance for determination of a quality measure. The respective analysis module is trained for the defined specific question and/or task.

Furthermore, it may also be that the at least one analysis module the at least one item of evaluation information by means of a hybrid approach, e.g. a combination of an algorithm, comprising an artificial neural network and a rule-based algorithm. It has been found here that this type of hybrid approach for analysis modules offers particularly high reliability of the evaluation information and/or the assistance information. In such a hybrid approach, a first subtask of the analysis module may be solved by an artificial neural network, for example a deep learning model, and a second subtask of the analysis module by means of a rule-based algorithm.

It may also be that for determination of assistance information, two or more analysis modules are used and/or required. At the same time, the different analysis modules may also have different approaches for determination of evaluation information. In an embodiment, each of the at least two analysis modules determines an item of evaluation information independently of the further analysis modules. The assistance information is then generated from all the evaluation information available. The assistance information may, for example, be generated by means of artificial intelligence (AI). At the same time, it may be that for generation of the assistance information the AI weights the individual items of evaluation information differently. Weighting of the individual evaluation information may be dependent upon the type of measurement program, e.g. the individual measurement steps, and/or the type of analysis module.

In an embodiment, a machine learning-based algorithm has been trained to detect certain characteristics and/or a certain pattern in the input data to be analyzed in relation to the question to be clarified. For the training, training data sets may be used, whose input data, e.g. magnetic resonance data, for example k-space data and/or magnetic resonance raw data, and/or reconstructed image data and/or further measurement information, has already been evaluated with regard to a defined clinical question. In an embodiment, training data sets from different training patients are available.

In this way, a rapid and robust assessment of the defined specific question and/or task by means of the at least one analysis module during execution of the measurement program may be provided for a user. In an embodiment, the user may be advantageously supported and manual and/or subjective errors in the assessment of the defined specific question and/or task reduced and/or prevented. Furthermore, rapid support may also be provided for complex questions.

In an advantageous development of the inventive method, it may be provided that based on the at least one provided evaluation information of the at least one analysis module at least one suggestion for at least one measurement step of the measurement program is determined by means of a recommendation module of the assistance system and the at least one suggestion for the at least one measurement step is provided. The assistance system may comprise multiple recommendation modules, the individual recommendation modules being designed to determine at least one suggestion for a respective defined and/or specific question. The determination of the at least one suggestion is dependent upon input data of the at least one recommendation module, the input data provided by the at least one analysis module comprising evaluation information. The at least one suggestion of the respective recommendation module comprises output data of the recommendation module. If evaluation information from different analysis modules is present different recommendation modules for determining at least one suggestion in each case may also be provided, the different recommendation modules determining a suggestion according to different questions. Furthermore, a single recommendation module may determine a suggestion based on evaluation information from two or more analysis modules. For measurement programs, with which a decision must be defined by the user for the further measurement process depending on the acquired magnetic resonance data during the execution of the measurement program, the suggestion may also specify a recommendation for the further measurement process.

Data transfer between the analysis modules and the recommendation modules may take place by means of the transaction manager of the assistance system. For example, the transaction manager may provide evaluation information from analysis modules to recommendation modules. In an embodiment, the transaction manager is designed to provide the evaluation information of a certain and/or special analysis module to a recommendation module, designed to determine at least one suggestion based on the evaluation information of the certain and/or special analysis module. The individual analysis modules and/or the individual recommendation modules may in each case have a corresponding interface, communicating with the transaction manager.

Selection and/or configuration of the at least one recommendation module may take place by means of the configuration user interface of the assistance system. At the same time, the at least one recommendation module may be directly selected and configured for at least one measurement step. Furthermore, when selecting at least one analysis module at least one recommendation module may be suggested to the user for selection. Moreover, it may also be the case that the user selects at least one recommendation module on the configuration user interface of the assistance system and receives a suggestion for at least one analysis module for selection, the at least one analysis module being able to provide the input data for the at least one recommendation module.

This embodiment of the disclosure has the advantage that during execution of the measurement program a user may be provided directly with a suggestion for improving and/or remedying and/or preventing a problem detected by an analysis module and/or an abnormality detected by an analysis module. Consequently, the user is not only made aware of a problem and/or an abnormality, but is also provided immediately with a suggestion for correcting the problem and/or analyzing the abnormality. This enables inexperienced users in particular to successfully complete the measurement program, even with problem patients, and to provide relevant image data for a diagnosis.

Alternatively or in addition, it may be provided that the at least one recommendation module together with at least one analysis module is coordinated with a defined clinical question and/or a defined technical question of the measurement program. The at least one suggestion of the at least one recommendation module may comprise a suggestion for problem solving for the at least one measurement step, provided that the provided evaluation information has detected a problem and/or a critical state in the at least one measurement step. For example, at least one technical analysis module and at least one technical recommendation module for answering a defined technical question, which may be related to the selected measurement program, may be provided. A technical recommendation module may be designed to process at least one item of evaluation information of at least one technical analysis module. For example, in the case of a detected problem and/or a detected critical state of an incorrectly positioned local high frequency coil by means of a technical analysis module the technical recommendation module may provide a user with a suggestion for correct positioning of the local high frequency coil. Consequently, a user may be provided with a specific suggestion for correcting a specific problem that is coordinated with the problem.

Furthermore, at least one quality analysis module and/or at least one clinical analysis module, together with a clinical recommendation module for answering a clinical question that may be related to the selected measurement program, may be proposed. A clinical recommendation module is designed to process at least one item of evaluation information of at least one clinical analysis module and/or at least one quality analysis module. For example, in the case of a detected problem and/or a detected critical state of an undesired patient motion during magnetic resonance data acquisition, the technical recommendation module may suggest a repeat of the measurement step and/or an adaptation of at least one measurement parameter and/or a selection of an alternative measurement sequence for this measurement step, etc. Consequently, a user may be provided with a specific technical suggestion for correcting a specific technical problem that is coordinated with the problem.

Alternatively or in addition, it may be provided that for determining the suggestion the at least one recommendation module comprises at least one suggestion coordinated with a detected problem and/or a detected abnormality, e.g. with multiple suggestions coordinated with the detected problem and/or the detected abnormality. In an embodiment, a recommendation module comprises multiple suggestions, available for correcting a problem and/or an abnormality or also for correcting multiple problems and/or multiple abnormalities. The recommendation module then selects the suggestion for correcting a detected problem and/or a detected abnormality, which based on the provided evaluation information offers the best solution for the detected problem and/or the detected abnormality. For example, for this purpose the at least one recommendation module may have a rule-based algorithm, which based on the input data, e.g. at least one item of evaluation information of at least one analysis module, determines and provides a suggestion and/or a recommendation for further action. This enables, for different detected problems and/or complications and/or different detected abnormalities, different solutions to also be provided by means of the at least one recommendation module and/or to be offered to a user.

Alternatively or in addition, it may be provided that the at least one recommendation module comprises multiple available suggestions, the multiple suggestions being stored in a database. The recommendation module may comprise the database. This enables rapid retrieval of the suggestions for output to the user. Furthermore, it is also conceivable that multiple recommendation modules are available to the database, it being possible for different recommendation modules to be linked and/or connected to the same suggestion in the database. For example, different recommendation modules for different clinical questions, but for which the same problem was detected, for example an undesired patient motion, may provide similar or identical suggestions for the user.

Alternatively or in addition, it may be provided that the at least one recommendation module determines extended assistance information, the extended assistance information comprising the at least one determined suggestion, and/or status information of the at least one recommendation module. The status information of the recommendation module may display to the user what type the recommendation module is, for example whether a quality recommendation module and/or a technical recommendation module and/or a clinical recommendation module is available and/or has been selected for the assessment of the measurement steps. Furthermore, the status information may also show the user if an analysis by means of the recommendation module is currently being carried out. Furthermore, the status information may show the user if the analysis by means of the recommendation module has been successfully completed and a result, e.g. a suggestion, is available. At the same time, the status information may also display to the user if further steps are necessary for correcting a problem and/or for clarifying an abnormality. Furthermore, the status information may display to the user if the analysis by means of the recommendation module could not be carried out. In an embodiment, the status information of the recommendation module may be displayed by corresponding symbols, which advantageously are arranged at a defined position on the user interface. This allows the user to be provided with rapid overview of the recommendation module. Furthermore, apart from the status information a possible suggested solution may also be displayed directly to the user which may contribute towards correcting a possible problem and/or clarifying an abnormality.

Alternatively or in addition, it may be provided that the extended assistance information is output to a user via a user interface for controlling and/or monitoring the measurement program. The user interface for controlling and/or monitoring the measurement program may e.g. display the individual measurement steps of the measurement program. At the same time, for the individual measurement steps the recommendation modules activated and/or selected for the respective measurement step may also be displayed. In an embodiment, the extended assistance information for the respective measurement step is displayed in the user interface. In an embodiment, the extended assistance information is displayed and/or output at a defined position on the user interface. In this way, simple and rapid transmission of the extended assistance information to the user may take place. In an embodiment, the user may concentrate on a single user interface and receive all information available for the measurement program including the extended assistance information.

Alternatively or in addition, it may be provided that the assistance system also comprises its own user interface for output of information, e.g. the extended assistance information. Such a user interface may for example comprise an assistance user interface, which apart from the assistance information may also inform the user which analysis module and/or recommendation module is currently being executed and for which measurement step this was selected.

Alternatively or in addition, it may be provided that following execution of a suggestion, feedback information from a user regarding the success of the suggestion may be entered. By means of the feedback the success of the suggested solutions may be determined. If the at least one recommendation module and/or the assistance system comprises a self-learning algorithm, a sequence of the available suggestions may be adapted according to success rate, so that a user in the first instance receives the most promising suggestion for improving and/or remedying a detected problem and/or the most promising suggestion for clarifying an abnormality.

In an advantageous development of the method it may be provided that the assistance system for execution of the at least one suggestion is designed for the at least one measurement step. In an embodiment, the assistance system comprises a control unit that controls an execution of the at least one suggestion for the at least one measurement step. Furthermore, the at least one recommendation module may also be designed for an execution of the at least one suggestion. Here, also the at least one recommendation module also comprises a control unit that controls an execution of the at least one suggestion for the at least one measurement step. At the same time, the control unit controls the hardware components for execution of the at least one measurement step according to the at least one suggestion. At the same time, such a suggestion may comprise a repetition of the measurement step and/or an adaptation of at least one measurement parameter during a repetition of the measurement step and/or an execution of an alternative measurement step with an alternative measurement sequence to the measurement step up to that point, etc.

The control unit of the assistance system comprises at least one calculation module and/or a processor, the control unit being designed for execution of at least one of the suggestions suggested by the recommendation module. So the control unit may be designed to execute computer-readable instructions, to execute the suggestion. In an embodiment, the control unit comprises a storage unit, with computer-readable information being stored on the storage unit, the control unit being designed to load the computer-readable information from the storage unit and to execute the computer-readable information, to execute the suggestion. In this way, the control unit is designed to execute at least one of the suggestions suggested by the recommendation module.

The components of the control unit may for the most part be designed in the form of software components. Essentially, however, these components may also in part, e.g. if particularly rapid calculations are involved, be implemented in the form of software-supported hardware components, for example FPGAs or the like. The required interfaces may also, for example, be designed as software interfaces, for example if it is only a matter of transferring data from other software components. However, they may also be designed as hardware-based interfaces that are controlled by suitable software. Of course, it is also conceivable for several of the stated components to be implemented in the form of an individual software components or software-supported hardware components.

This embodiment of the disclosure has the advantage that an easy and rapid implementation of the at least one suggestion suggested by the recommendation module may be provided for a user. For instance, in this way a correct implementation and/or execution of the at least one suggestion may be ensured.

Alternatively or in addition, it may be provided that the assistance system is designed for an execution of the at least one suggestion for the at least one measurement step in a semi-automatic execution mode or in a fully-automatic execution mode. In the semi-automatic execution mode the execution takes place only after the user has made a confirmation entry. This confirmation entry is a trigger for the assistance system for an automatic execution of the at least one suggestion. However, if the user rejects the automatic execution of the suggestion by the assistance system, it is up to the user to execute or not execute the suggestion manually. In the automatic execution mode the at least one suggestion is executed automatically without asking the user first. Furthermore, it may be provided that after an execution of a suggestion suggested by a recommendation module by means of the semi-automatic or the automatic execution mode, information on the execution is output to the user. Thus, a simple and rapid implementation of the at least one suggestion suggested by the recommendation module may be provided for a user. For instance, in this way a correct implementation and/or execution of the at least one suggestion may be ensured.

Alternatively or in addition, it may be provided that the user for each measurement step of the measurement program may select the execution mode for execution of a suggestion. In an embodiment, selection of the execution mode for each of the measurement steps of the measurement program takes place by means of the configuration user interface. The assistance system enables the user, for each measurement step, to specify an individual selection of the execution mode. At the same time, for individual measurement steps, that are of special interest to the user, the user may select the semi-automatic execution mode and thereby maintain control over possible suggestions to be executed.

In an advantageous development of the method, it may be provided that the measurement program comprises multiple measurement steps and, for at least one of the multiple measurement steps, support by means of the assistance system is selectable for a user. At the same time, for particularly critical measurement steps, the user may select support by means of the assistance system. In an embodiment, the selection of support for at least one of the measurement steps takes place by means of the configuration user interface. It may be advantageous for a user to be able to select support by means of the assistance system for each of the several measurement steps in the measurement program. The provision of a selection of at least one recommendation module and/or at least one analysis module for the at least one measurement step may e.g. take place by means of a configuration user interface of the assistance system. In an embodiment, the user may select and set individual support for each of the multiple measurement steps. For example, users with many years' experience of magnetic resonance examinations may select support only for particularly critical measurement steps of the measurement program. On the other hand, users with less experience of magnetic resonance examinations may select support for all measurement steps of a measurement program. For example, by means of the configuration user interface a user may select for which measurement steps they want to have support, e.g. which analysis modules and/or recommendation modules they want for a particular measurement step. Furthermore, the user may also configure the individual modules, e.g. the individual analysis modules and/or recommendation modules, if they only want to have feedback and/or information on the individual measurement steps or also a recommendation for the further action of the measurement program.

Alternatively or in addition, it may be provided that for at least one of the multiple measurement steps of the measurement program at least one analysis module and/or at least one recommendation module is assigned and is selectable by a user. In an embodiment, the assistance system assigns at least one analysis module and/or at least one recommendation module to the at least one measurement step of the measurement program, the assignment being made using the configuration user interface of the assistance system. The at least one analysis module and/or the at least one recommendation module may e.g. be coordinated with a clinical and/or diagnostic question of the measurement program and/or of the at least one measurement step. If, for example, the measurement program comprises a head examination of the patient, the at least one assigned analysis module is designed for analysis of head image data and/or further measurement information concerning the head examination. The at least one recommendation module is designed for determining a suggestion concerning a head examination. If, on the other hand, the measurement program for example comprises a cardiac examination of the patient, the at least one assigned analysis module is designed for analysis of heart image data and/or further measurement information concerning the cardiac examination. The at least one recommendation module is designed for determining a suggestion concerning a cardiac examination. In this way, advantageous support for a user in the execution of the measurement program may be made available. In an embodiment, the user may already be advantageously supported in the selection of at least one analysis module and/or at least one recommendation module by the assistance system. Furthermore, a selection of analysis modules and/or recommendation modules, that are not suitable for supporting the selected measurement program, may be advantageously reduced and/or prevented.

Alternatively or in addition, it may be provided that for at least one of the multiple measurement steps of the measurement program at least one analysis module is selectable by a user, the selection of the at least one analysis module comprising a suggestion for a selection of at least one recommendation module. For example, such linking between an analysis module and at least one recommendation module may also be established by means of a configuration user interface of the assistance system, which upon selection of the at least one analysis module selects the output data provided by the at least one analysis module, e.g. the at least one item of evaluation information, selects and/or provides for selection a recommendation module designed for processing the output data provided by the analysis module. In an embodiment, the selection of the at least one recommendation module with the at least one selected analysis module is also dependent upon an examination-relevant and/or measurement program-relevant context. This allows advantageous support to be made available for a user. In an embodiment, a recommendation module may be suggested to the user, which is compatible with the selected analysis module. Moreover, a selection of a recommendation module, that is not suitable for supporting the selected measurement program or is incompatible with the selected analysis module, may advantageously be reduced and/or prevented.

Alternatively or in addition, it may be provided that for at least one of the multiple measurement steps of the measurement program at least one recommendation module is selectable by a user, the at least one recommendation module requires a data input, the data input being automatically linked with at least one analysis module. The at least one recommendation module may be designed for determining suggestions for a defined magnetic resonance examination and/or a defined measurement program and/or a defined clinical and/or diagnostic question. The input data required for determining suggestions is provided by the automatic linking and/or selection of at least one analysis module during an execution of the measurement program. For example, such linking between at least one recommendation module and at least one analysis module may also be established by means of a configuration user interface of the assistance system, which upon selection of the at least one recommendation module with the help of the necessary input data for the at least one recommendation module and with the help of the clinical question selects and/or activates the analysis modules linked with the necessary input data. If the at least one recommendation module is designed for determining suggestions for a complex magnetic resonance examination, it may also be that multiple analysis modules are automatically linked with the at least one recommendation module and automatically selected together with the selection of the recommendation module. For example, a recommendation module, designed for cardiac examinations, may be linked with an analysis module, that analyzes an ECG monitoring of the patient, an analysis module, that analyzes the breathing monitoring of the patient, and an analysis module, that analyzes the acquired heart image data. For comprehensive monitoring and supporting the cardiac examination the recommendation module requires the evaluation information of the three analysis modules as input data. In an embodiment, the recommendation module, depending on the different input data, is also designed for different possible problems and/or for different abnormalities, to determine suggestions and provide these to the user. This allows advantageous support to be made available for a user. In this way, it may be ensured that all necessary input data for comprehensive support and/or monitoring of the measurement program is available.

In an advantageous development of the method it may be provided that the measurement program has multiple measurement steps, and the multiple measurement steps of the measurement program are executed sequentially, with for at least one measurement step support by means of the assistance system having been selected, a measurement step following the at least one measurement step, for which the support was selected, is only started if for the at least one measurement step, for which the support was selected, the support by means of the assistance system is complete. This allows a simple type of the support to be implemented. In an embodiment, for inexperienced users an advantageous clarity for the individual measurement steps and for the displayed information and/or suggestions to support the respective measurement step is provided. Consequently a possible confusion for a user of measures for individual measurement steps may be reduced and/or prevented.

In an advantageous development of the method it may be provided that the measurement program comprises multiple measurement steps and for each measurement step of the measurement program, for which support by means of the assistance system has been selected, the support is carried out by means of the assistance system in a quasi-real time mode or in a real-time mode. The quasi-real time mode is designed so that e.g. predefined blocks of partially acquired data may even be transferred to the corresponding analysis module for analysis during the execution of the measurement steps. In this way, data may already be analyzed and the user supported while the measurement step is still being carried out. This enables direct feedback of possible problems and/or possible abnormalities to the user. Furthermore, this also allows the measurement program to be considerably accelerated and measurement time and/or examination time to be saved. For example, an analysis module may analyze segments and/or blocks of a respiratory signal of the patient, being acquired in the measurement step currently being executed, regarding the question, of whether the patient is complying with the breath holding instruction. In this way it may be constantly monitored, e.g. even during the current measurement steps, if the patient is meeting the conditions for acquisition of magnetic resonance data. Possible suggestions, for example warnings and/or workflow recommendations, of a recommendation module, based on the evaluation information of the analysis module, may also be executed during the measurement step.

The real time mode may be e.g. designed so that acquired data is continuously transferred in real time to the corresponding and/or selected analysis modules. The analysis modules determine in real time evaluation information with a continuous data stream, while the measurement step is being executed. For example, it is advantageous to monitor patient motion detection during motion-critical measurement steps continuously and real time. This allows data analysis by the analysis modules to be accelerated. The user may also be offered support, as soon as a critical state of an analysis module is detected. In an embodiment, support may be displayed to the user at any time during execution of the measurement steps.

Selection of an execution mode for support by the assistance system, e.g. if a simple execution mode or a quasi-real time mode or a real time mode is required, may e.g. take place during selection and/or configuration of the individual modules by means of the configuration user interfaces.

In an advantageous development of the method it may be provided that an exchange and/or the provision of the magnetic resonance data and/or the image data and/or the further measurement information and/or the at least one item of evaluation information and/or the at least one item of assistance information and/or at least one item of extended assistance information of a recommendation module takes place by means of a transaction manager of the assistance system. At the same time, the transaction manager also controls data transfer between individual analysis modules and individual recommendation modules, so that for each analysis module and/or recommendation module the correct and/or relevant input data is present for further processing. For this, the transaction manager may also have a processing unit and/or a control unit. This allows advantageous support to be made available for a user. In an embodiment, it may be ensured that the functionality of the individual modules may be guaranteed to support the user during the execution of the measurement program. In an embodiment, the input data required for the functioning of all modules required to support the user may be provided in this way.

In an advantageous development of the method it may be provided that the assistance system comprises a configuration user interface, the configuration user interface being designed to configure a selection of at least one analysis module and/or at least one recommendation module for at least one measurement step. The configuration user interface may e.g. be designed for a measurement program and/or individual measurement steps of a measurement program to provide a user with a selection of available modules, e.g. analysis modules and/or recommendation modules, for selection and/or configuration. At the same time, the user may specify with which analysis module and/or recommendation module at which position in the measurement program support is to be given or also for which measurement step support with at least one analysis module and/or at least one recommendation module is to be given. Furthermore, with some analysis modules and/or recommendation modules the user may also have the opportunity, by means of the configuration user interface to select a data type and/or a data format of input data for the selected module and/or adapt the corresponding module to a measurement step. Furthermore, by means of the configuration user interface linking of multiple analysis modules may be configured and/or selected by a user. Selection of at least one module, e.g. at least one recommendation module and/or at least one analysis module, and/or a combination of modules may also be suggested to the user for at least one measurement step by means of the configuration user interface. The user may also configure the individual modules, e.g. the individual analysis modules and/or recommendation modules, if they only want to have feedback and/or information on the individual measurement steps or also a recommendation for the further action of the measurement program.

This allows easy and rapid selection for a user of a module, e.g. at least one analysis module and/or at least one recommendation module. This embodiment of the disclosure also enables inexperienced and/or novice users to easily use the analysis module and/or recommendation module during execution of a measurement program. Furthermore, it may also be ensured in this way that a data set, e.g. an image data set, is available after the end of the measurement program, from which a diagnosis may be made.

Alternatively or in addition, it may be provided that the configuration user interface is designed to specify a data format of input data of the analysis modules. The input data of the analysis modules comprises the provided magnetic resonance data and/or the provided reconstructed image data and/or the provided further measurement information. This means that the input data may also be made available for different analysis modules for analysis without having to prepare the input data for the respective analysis module beforehand. This also enables a simple exchange of analysis modules for an analysis of the provided input data.

Alternatively or in addition, it may be provided that the input data comprises the provided magnetic resonance data, the provided magnetic resonance data comprising k-space data and/or magnetic resonance raw data, and/or reconstructed image data, the magnetic resonance data and/or the image data comprising a DICOM format and/or an ISMRMRD format. DICOM (Digital Imaging and Communications in Medicine) comprises an open standard for exchange and/or storage of medical image data. This medical image data may for example comprise digital images, additional information such as segmentations, surface definitions, or image registrations. By means of the DICOM format, both the format for storage of the medical image data and the communication protocol for its exchange are standardized. The ISMRMRD format is a common MR raw data format that facilitates cooperation, shared use and exchange of data, and data processing tools. In this way, advantageously uniform data formats are used for the provided magnetic resonance data and/or image data, which are already standard in the processing and/or analysis of magnetic resonance data and/or medical image data, thus enabling simple implementation of the analysis modules.

Alternatively or in addition, it may be provided that the input data comprises the provided further measurement information, the provided further measurement information comprising a data format, that is dependent upon a data type of the provided further measurement information. The further measurement information may e.g. comprise physiological data of the patient and/or further patient data and/or technical data. The physiological data may comprise a respiratory signal and/or an ECG signal and/or further physiological data. The physiological data may e.g. comprise an ISMRMRD format or further public community standard data formats. Furthermore, proprietary data formats are also conceivable. The further patient data may comprise patient age and/or height of the patient and/or weight of the patient and so on, the further patient data comprising data necessary and/or important for the execution of the measurement program. The further patient data may e.g. comprise units, such as meters, KG, years and so on. The technical data may comprise a hardware parameter such as a temperature and/or plug contact of a coil connector and so on. The technical data may e.g. comprise an ISMRMRD format or further public community standard data formats. Furthermore, proprietary data formats are also conceivable. Furthermore, the technical data may also comprise a temperature unit. In this way, broad application of the provided further measurement information may be ensured. Furthermore, different analysis modules may also access the further patient data and thus in the standardized data formats, without the input data for the respective analysis module and/or the analysis module having to be prepared and/or adapted to the input data beforehand.

In an advantageous development of the method, it may be provided that the at least one analysis module and/or the at least one recommendation module generates output data, the output data comprising at least partially a defined output data format. Specification of a defined output data format may e.g. take place by means of the configuration user interface. The output data with the defined and/or specific output data format may e.g. comprise the evaluation information and/or the status information of the analysis modules. Furthermore, the output data with the defined and/or specific output data format may also comprise the status information of the recommendation modules. This enables simple further processing of the provided output data. In an embodiment, different recommendation modules may thus access the output data provided by an analysis module without the need for a complex adaptation of the provided output data and/or the recommendation modules. Furthermore, an exchange of modules may be simplified, since a data format of both input data and output data is standardized for the individual modules.

Alternatively or in addition, it may be provided that the output data of the analysis modules comprises defined data classes, categorization of the output data in a data class comprising a semantic test of the analysis module question to be clarified. This enables a simple transfer of the data to further modules.

The defined data classes comprise a classification of the output data of the analysis modules. For classification of the output data of the analysis modules a traffic light classifier, a binary classifier, a multi-class classifier, and a multi-class multi-label classifier are available. Furthermore, in an alternative development of the disclosure, classification classes which the person skilled in the art deems useful may also be available. For categorization of the output data into the individual data classes the initial question firstly arises of whether the clinical question and/or the analysis of the at least one analysis module may be formulated as a binary classification from 1 to N. If the answer to this initial question is “Yes”, then both the traffic light classifier and the binary classifier classes are available. For further subdivision the condition may be analyzed as to whether in addition the output options of the analysis module may be answered with “Yes” or “No” or with “Good” or “Poor”. In such a case the output data is assigned the traffic light classification. However, if the condition is not met the output data is assigned the binary classification.

However, if the answer to the initial question is a “No”, then both the multi-class classifier and multi-class multi-label classifier classes are available. With the multi-class classifier the data may be subdivided into three or more categories. If each instance and/or category comprises precisely one defined value, the output data is assigned the multi-class classifier. However, if multiple statements or values are possible for each instance and/or category, the output data is assigned the multi-class multi-label classifier.

With a traffic light classification or a binary classification the values “0”, “1” and “−1” are available as output values. The value “0” indicates that no abnormalities or problems have been detected by means of the analysis module and the measurement program may be continued as planned. In an embodiment, no actions by the user for correcting a critical situation are necessary. The value “1” indicates that critical states or problems or abnormalities have been detected by means of the analysis module. This often requires interaction on the part of the user for correcting the detected critical state or the detected problem or the detected abnormality. The value “−1” indicates that there is no clear result of the analysis. A positive or negative result could not be established with sufficient certainty. The user is often alerted to a possible problem or an abnormality and recommended to check the data manually and make their own decision.

With a multi-class classifier the values “(1, 0, 0, . . . , 0)”, “(0, 1, 0, . . . , 0)” to “(0, 0, 0, . . . , 1)” and “−1” are available as output data. The values “(1, 0, 0, . . . , 0)”, “(0, 1, 0, . . . , 0)” to “(0, 0, 0, . . . , 1)” indicate that in each case a specific case has been detected by means of the analysis module. The value “−1” indicates that there is no clear result of the analysis.

With a multi-class multi-label classifier the values “(1, 0, 0, . . . , 0)”, “(1, 1, 0, . . . , 0)” to “(1, 1, 1, . . . , 1)” and “−1” are available as output data. The values “(1, 0, 0, . . . , 0)”, “(1, 1, 0, . . . , 0)” to “(1, 1, 1, . . . , 1)” indicate that in each case a specific case has been detected by means of the analysis module. The value “−1” indicates that there is no clear result of the analysis.

Moreover, the disclosure is based on an assistance system designed to execute the method for supporting and/or assisting a user when executing a measurement program during magnetic resonance data acquisition, the analysis module having a transaction manager and at least one analysis module. In an embodiment, the transaction manager is designed for data exchange between the at least one analysis module and other units and/or modules. In an embodiment, the transaction manager is designed to provide input data, e.g. magnetic resonance data and/or image data and/or further measurement information, for the at least one analysis module. In an embodiment, the transaction manager is also designed to provide the output data of the at least one analysis module to further units and/or modules. At the same time, the output data of a user interface may be made available for output to a user. Furthermore, the output data may also be made available for further processing to further modules of the assistance system. The output data may also comprise an indication, e.g. assistance information, to the user, of a problem detected by means of the at least one analysis module and/or of a critical state detected by means of the at least one analysis module and/or of an abnormality detected by means of the at least one analysis module.

The at least one analysis module is designed for analysis of input data, e.g. the provided magnetic resonance data and/or the provided reconstructed image data and/or the provided further measurement information. At the same time, the at least one analysis module is designed to analyze the input data for abnormalities and/or irregularities.

By means of the assistance system, a user may during a magnetic resonance examination, e.g. execution of the measurement program, be made aware or given an indication of abnormalities and/or problems and/or critical states with the magnetic resonance data acquisition. Consequently, the user receives direct feedback and may decide on the basis of the information, e.g. the assistance information, whether they want to take measures to correct the errors or problems or prevent the occurrence of probable problems, for example repeat individual measurement steps of the measurement program or change recording parameters. For example, the user may also change the time for which the patient holds their breath if they are having problems holding their breath for a long time. Moreover, the user may also select an alternative measurement sequence for the measurement step. Furthermore, the user may decide that despite the quality of the image data being poor, it is nevertheless adequate for assessing the clinical or diagnostic question. Furthermore, based on the information provided by the assistance system, the user may also decide whether they want to take further measures to clarify a detected abnormality, such as inserting additional measurement steps focusing on the area with the detected abnormality.

By immediately informing the user, e.g. by the output of the assistance information while the measurement program is running, effort, such as examination effort, may be reduced for the patient and the user. If, for example, the acquired magnetic resonance data and/or image data reconstructed from the magnetic resonance data is unsuitable for a diagnostic assessment, the measurement step or multiple measurement steps may be repeated and/or executed with changes. A repeat only after days or weeks, once the magnetic resonance data and/or image data has been analyzed and a repeat appointment found for the patient, may thereby be avoided. For example, only the relevant, e.g. only the defective and/or flawed measurement steps, need be repeated and not the entire measurement program.

The advantages of the assistance system essentially correspond to the advantages of the method for supporting and/or assisting a user when executing a measurement program during magnetic resonance data acquisition, explained in detail above. The features, advantages, or alternative embodiments mentioned here may also be transferred to the other claimed subject matters, and vice versa.

In an advantageous development of the assistance system, it may be provided that the assistance system comprises at least one recommendation module. The at least one recommendation module is designed to determine based on input data a suggestion for the further action of the measurement program. The input data of the at least one recommendation module may comprise output data determined by the at least one analysis module, provided by means of the transaction manager to the at least one recommendation module. In an embodiment, the suggestion determined by the at least one recommendation module is provided for output to a user of a user interface, the provision of the suggestion determined taking place by means of the transaction manager.

This embodiment of the disclosure has the advantage that during execution of the measurement program a user may be provided directly with a suggestion for improving and/or remedying a problem detected by an analysis module and/or a critical state detected by an analysis module and/or an abnormality detected by an analysis module.

Consequently, the user is not only made aware of a problem and/or a critical state and/or an abnormality, but also receives immediately a suggestion for correcting the problem and/or the critical state and/or for analyzing the abnormality. This enables inexperienced users to successfully complete the measurement program, even with problem patients, and to provide relevant image data for a diagnosis.

In an advantageous development of the inventive assistance system, it may be provided that the transaction manager comprises a communication interface for data exchange of the at least one analysis module and/or the at least one recommendation module. In this way simple communication between the individual modules, e.g. between the analysis modules and/or the recommendation modules, of the assistance system may be enabled.

In an advantageous development of the assistance system, it may be provided that the assistance system comprises a configuration user interface, the configuration user interface being designed to provide a selection of analysis modules and/or recommendation modules for a selected measurement program. In an embodiment, a selection of analysis modules and/or a recommendation module is provided for at least one measurement step of the selected measurement program. In an embodiment, the configuration user interface makes available a selection of analysis modules and/or recommendation modules, which are coordinated with the measurement program, e.g. with the at least one measurement step of the measurement program. At the same time, the configuration user interface may be designed so that upon selection of a module, e.g. an analysis module and/or a recommendation module, further modules compatible with the selected module, e.g. with the output data provided by the selected module or required input data, are automatically recommended to the user, said further modules also corresponding to an examination-relevant and/or measurement program-relevant context. Furthermore, such further modules may be automatically activated and/or selected by the configuration user interface if, for example, a functionality of the selected module requires this.

This allows easy and rapid selection for a user of a module, such as for instance at least one analysis module and/or at least one recommendation module. This embodiment of the disclosure also enables inexperienced and/or novice users to easily use the analysis module during execution of a measurement program. Furthermore, it may also be ensured in this way that a data set, e.g. an image data set, is available after the end of the measurement program, from which a diagnosis may be made.

In an advantageous development of the assistance system, it may be provided that the configuration user interface is integrated at least partially in a user interface for controlling and/or monitoring of the measurement program. At the same time, a selection of at least one module for at least one measurement step may take place via the user interface, it being possible to recall or click on the configuration user interface by way of the operator interface and to configure a type of support. Furthermore, the modules of the assistance system activated and/or selected for the measurement step, e.g. the analysis modules and/or the recommendation modules, may also be displayed for the individual measurement steps. In this way simple and rapid transmission of assistance system information to the user may take place. In an embodiment, the user may concentrate on a single user interface and receive all information available for the measurement program.

In an advantageous development of the assistance system, it may be provided that the configuration user interface specifies a data format for input data and/or output data of the at least one analysis module and/or of the at least one recommendation module. Thanks to a standardized data format different modules, e.g. analysis modules and/or recommendation modules, may access the provided data, without the data, e.g. input data, having to be prepared for the respective module. This enables a simple exchange of modules, e.g. of analysis modules and/or recommendation modules, within the assistance system.

In an advantageous development of the assistance system, it may be provided that the configuration user interface together with the transaction manager comprises a framework of the assistance system, the individual analysis modules and/or the individual recommendation modules being exchangeable within the framework. In an embodiment, the individual analysis modules and/or the individual recommendation modules, thanks to the specification of standardized data format for the input data and/or output data of the modules, e.g. the analysis modules and/or the recommendation modules are exchangeable in the framework. In this way, a particularly simple integration of further modules, e.g. analysis modules and/or recommendation modules, may take place. For example, in this way a number of modules available for selection may also be varied and/or extended.

In an advantageous development of the assistance system. it may be provided that the assistance system comprises a user interface for output of information, e.g. the assistance information and/or the extended item of assistance information. The user interface may comprise an assistance user interface, which apart from the assistance information, may also inform the user which analysis module is currently being executed and for which measurement step this was selected. In this way simple and rapid transmission of the assistance information to the user may take place. Alternatively, the user interface for output of information may also be integrated in a user interface for controlling and/or monitoring of the measurement program. The user interface for controlling and/or monitoring the measurement program may display the individual measurement steps of the measurement program. At the same time, for the individual measurement steps, the analysis modules activated and/or selected for the measurement step may also be displayed. In an embodiment, the assistance information for the respective measurement step is displayed in the user interface. Thus, the user may concentrate on a single user interface and receive all information available for the measurement program including the assistance information.

Moreover, the disclosure is based on a magnetic resonance device with an assistance system, the assistance system being designed to execute the method for supporting and/or assisting a user when executing a measurement program during magnetic resonance data acquisition.

The magnetic resonance device may comprise a medical and/or diagnostic magnetic resonance device, designed and/or configured for acquisition of medical and/or diagnostic image data, e.g. medical and/or diagnostic magnetic resonance image data, of a patient and/or object. The magnetic resonance device may comprise a magnet unit for acquisition of the medical and/or diagnostic magnetic resonance data. Here, the magnet unit comprises a base magnet, a gradient coil unit, and a high-frequency (e.g. radio frequency (RF)) antenna unit. The RF antenna unit may be permanently arranged and/or installed within the magnet unit. The magnet unit surrounds a patient receiving area of the magnetic resonance device. The patient receiving area may e.g. have a cylindrical design and is designed to receive the patient, e.g. the area of the patient to be examined, for a magnetic resonance examination.

The base magnet is designed to generate a homogeneous base magnet field with a defined magnetic field strength, for example with a magnetic field strength of 0.55 T or 1.5 T or 3 T or 7 T, and so on. In an embodiment, the base magnet is designed to generate a strong, constant and homogeneous base magnet field. The gradient system is designed to generate magnetic field gradients, used for spatial encoding during imaging. The RF antenna unit is designed for transmitting high-frequency pulses and/or excitation pulses for generation of magnetic resonance signals.

For a magnetic resonance examination the patient, e.g. the area of the patient to be examined, is positioned within the patient receiving area of magnetic resonance device. The Field of View (FOV) and/or an isocenter of the magnetic resonance device may e.g. be arranged within the patient receiving area. The FOV may comprise an acquisition area of the magnetic resonance device, within which the conditions exist for acquisition of medical image data, e.g. magnetic resonance image data, within the patient receiving area, such as a homogeneous base magnet field. The isocenter of the magnetic resonance device may e.g. comprise the area and/or point within the magnetic resonance device, that has the optimum and/or ideal conditions for the acquisition of medical image data, e.g. magnetic resonance image data. In an embodiment, the isocenter comprises the most homogeneous magnetic field region within the magnetic resonance device.

The magnetic resonance device may e.g. include the assistance system. At the same time, the assistance system may be fully or at least partially, e.g. individual modules of the assistance system, executed on a separate server, e.g. on computers separate from the system control unit. Furthermore, it is also conceivable for the assistance system to be fully or at least partially, e.g. individual modules of the assistance system, executed in a cloud and/or an edge device.

The advantages of the magnetic resonance device essentially correspond to the advantages of the method for supporting and/or assisting a user by means of an assistance system when executing a measurement program during magnetic resonance data acquisition, explained in detail above. The features, advantages, or alternative embodiments mentioned here may also be transferred to the other claimed items, and vice versa.

Moreover, the present disclosure is based on a computer program product, comprising a program and which may be loaded directly into a memory of a programmable control unit, with program means for executing a method for supporting and/or assisting a user by means of an assistance system when executing a measurement program during magnetic resonance data acquisition, when the program is executed in the control unit. At the same time, the computer program may require program resources, e.g. libraries and auxiliary functions, in order to implement the corresponding embodiments of the method. The computer program may comprise software with a source code that still needs to be compiled and bound or that only needs to be interpreted, or an executable software code that may be loaded into a corresponding processing unit for execution.

The computer program product may be loaded directly into a memory of a programmable processing unit, and has program code means for executing an inventive method, if the computer program product is executed in the processing unit. The computer program product can be a computer program or comprise a computer program. In this way, the method may be executed quickly, identically, repeatably, and robustly. The computer program product is configured so that it may execute any of the method steps by means of the processing unit. The processing unit may thus meet the respective criteria such as having a corresponding working memory, a corresponding graphic card, or a corresponding logic unit, so that the respective method steps may be executed efficiently. The computer program product is for example saved to a computer-readable medium or stored on a network or server, from where it may be loaded into the processor of a local processing unit, which may be directly linked to the magnetic resonance device or be designed to be part of. Control information of the computer program product may also be stored on an electronically-readable data carrier. The control information of the electronically-readable data carrier may be arranged in such a way that when the data carrier is used in a processing unit it executes an inventive method. Thus the computer program product may also constitute the electronically-readable data carrier. Examples of electronically-readable data carriers are a DVD, a magnetic tape, a hard disk or a USB stick, on which electronically-readable control information, e.g. software (see above), is stored. If this control information (software) is read from the data carrier and stored in a control and/or processing unit, all inventive embodiments of the method previously described in the introduction may be executed. Thus the disclosure may also be based on said computer-readable medium and/or said electronically-readable data carrier.

### BRIEF DESCRIPTION OF THE DRAWINGS

Further advantages, features and details of the disclosure result from the exemplary embodiment described below and based on the drawings, which show:

FIG. 1 illustrates a first exemplary embodiment of a method for supporting and/or assisting a user by means of an assistance system when executing a measurement program during magnetic resonance data acquisition;

FIG. 2 illustrates a first exemplary embodiment of an assistance system;

FIG. 3 illustrates a second exemplary embodiment of the method;

FIG. 4 illustrates a second exemplary embodiment of the assistance system;

FIG. 5 illustrates a further exemplary embodiment of the assistance system,

FIG. 6 illustrates a further exemplary embodiment of the assistance system,

FIG. 7 illustrates a further exemplary embodiment of the assistance system,

FIG. 8 illustrates a further exemplary embodiment of the assistance system,

FIG. 9 illustrates an example of the assistance system in a serial execution mode;

FIG. 10 illustrates an example assistance system in a quasi-real time mode;

FIG. 11 illustrates an example assistance system in a real time mode;

FIG. 12 illustrates an example of output data of an analysis module and/or of a recommendation module;

FIG. 13 illustrates an example user interface for selection of a module of the assistance system for a measurement step;

FIG. 14 illustrates an example user interface for displaying assistance information and/or a suggestion during execution of the measurement step;

FIG. 15 illustrates an example schematic representation of a magnetic resonance device with an assistance system.

### DETAILED DESCRIPTION OF THE DISCLOSURE

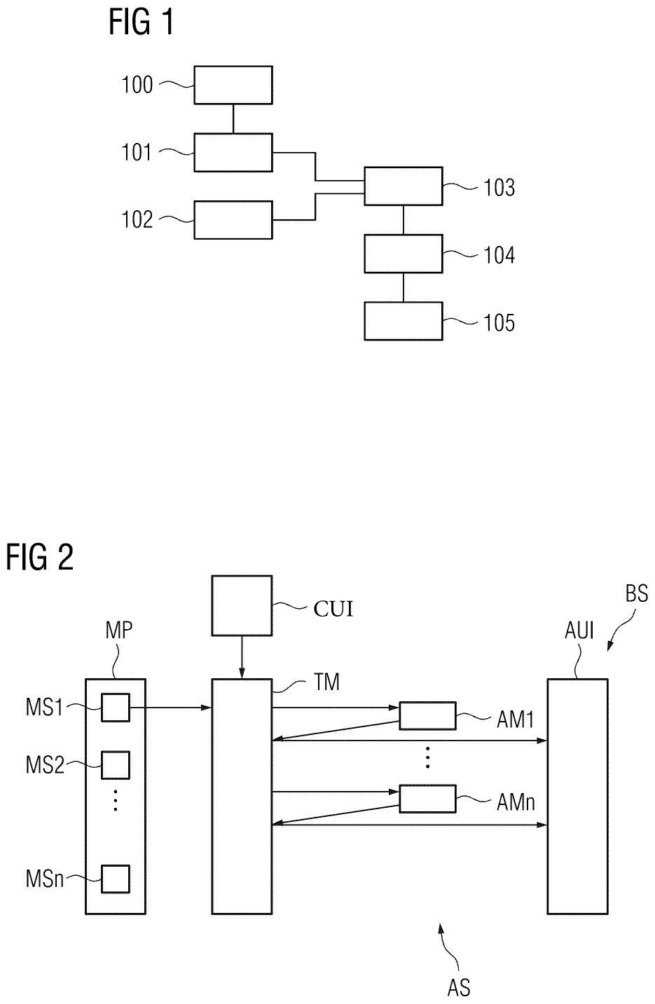

FIG. 1 shows a first exemplary embodiment of a computer-implemented method for supporting and/or assisting a user by means of an assistance system (AS) when executing a measurement program (MP) during magnetic resonance data acquisition. For execution of the method the assistance system (AS) has a transaction manager (TM) and at least one analysis module AM, with reference being made to FIG. 2 for the design of the assistance system AS. In an embodiment, the assistance system AS comprises multiple analysis modules AM, designed for analysis of different data concerning different questions.

The transaction manager TM may e.g. be designed for data exchange of the at least one analysis module AM. At the same time, the transaction manager TM may e.g. be designed for provision of input data for the at least one analysis module AM. Furthermore, the transaction manager TM may e.g. also be designed for provision of output data of the at least one analysis module AM, e.g. for further modules of the assistance system AS and/or further units, such as an output unit and/or a display unit of a user interface BS.

The assistance system AS also comprises a configuration user interface (CUI), that may communicate via the user interface with a user. Via the configuration user interface CUI a selection and/or a configuration of at least one analysis module AM by a user is provided. The transaction manager TM together with the CUI forms a frame and/or a framework of the assistance system AS, within which the individual modules, e.g. analysis modules AM, etc. In other words, the framework, e.g. the CUI, also specifies a format of input data and/or output data of the individual modules of the assistance system AS, and consequently also specifies a data format for communication of the individual modules.

For controlling the method for supporting and/or assisting a user by means of the assistance system AS when executing a measurement program MP during magnetic resonance data acquisition, the assistance system AS has a control unit and/or processing unit with corresponding software and/or corresponding programs, to control the method for supporting and/or assisting a user by means of the assistance system AS when executing a measurement program MP during magnetic resonance data acquisition.

In a first method step 100 of the method selection of a measurement program MP takes place for acquisition of magnetic resonance data with a defined diagnostic question. Together with the measurement program MP, the user may also select support by means of the assistance system AS and, consequently, at least one analysis module AM of the assistance system AM, designed for analysis of data during execution of the measurement program MP. At the same time, by selecting the measurement program MP a selection of analysis modules AM already coordinated with the clinical and/or diagnostic question of the measurement program MP and/or assigned selection may be offered to the user for selection. The measurement program MP comprises multiple measurement steps MS1, MS2, . . . , MSn, with for at least one measurement step MSi by means of the assistance system AS and, consequently, at least one analysis module AM of the assistance system AS support being selectable. Furthermore, for multiple measurement steps MS1, MS2, . . . , MSn of the measurement program MP support by means of the assistance system AS and consequently at least one analysis module AM of the assistance system AS may be selectable. At the same time, different analysis modules AM1, AM2, . . . , AMn for different measurement steps MS1, MS2, . . . , MSn of the measurement program MP may also be available and/or offered to the user for selection. The selection of the measurement program MP and also the selection of the at least one analysis module AM is made by a user via the configuration user interface CUI at the user interface BS. The user interface BS may be included in a magnetic resonance device.

Then, in a second method step 101 execution of the selected measurement program MP takes place, execution of the measurement program MP comprising acquisition of magnetic resonance data. During the acquisition of the magnetic resonance data in this second method step 101, image data is directly reconstructed from the magnetic resonance data. Reconstruction of the image data takes place via a reconstruction unit. The reconstruction unit may be included in the measurement program MP or form a separate unit to the measurement program MP. Furthermore, in this second method step 101 the magnetic resonance data and the reconstructed image data are provided. At the same time, the magnetic resonance data is provided in the form of k-space data and/or magnetic resonance raw data. Provision of the magnetic resonance data and/or the reconstructed image data take place by means of the transaction manager RM of the assistance system AS. The provided magnetic resonance data and/or provided reconstructed image data is provided as input data for the analysis modules AM1, AM2, . . . , AMn, a data format of the provided magnetic resonance data and/or the provided reconstructed image data comprising a DICOM format and/or an ISMRMRD format.

In a further third method step 102, further measurement information is also acquired and provided. The further measurement information may further data acquired during the execution of the measurement program MP, and consequently at the same time as execution of the measurement program MP, from the patient and/or hardware performing the magnetic resonance measurement. This further measurement information may, for example, comprise physiological data of the patient, e.g. a respiratory signal and/or an ECG signal and/or further physiological data. Furthermore, the further measurement information may also comprise data and/or parameters of a hardware configuration to be monitored of, for example, a local high frequency coil and so on. Furthermore, the further measurement information may also comprise further patient data, already acquired during registration of the patient for a magnetic resonance examination and consequently prior to the execution of the measurement program MP. Such further patient data may, for example, comprise the age and/or weight and/or height of the patient and so on.

In a further fourth method step 103, at least one item of evaluation information is determined by means of the at least one analysis module AM of the assistance system AS as a function of input data, provided to the at least one analysis module AS by means of the transaction manager TM. The input data of the at least one analysis module AM comprises the provided magnetic resonance data and/or the provided reconstructed image data and/or the provided further measurement information. In an embodiment, the at least one item of evaluation information is determined as a function of the magnetic resonance data and/or the reconstructed image data and/or the further measurement information. At the same time, by means of the configuration user interface CUI, a data format of the input data of the at least one analysis module AM is specified. The provided magnetic resonance data and/or the reconstructed image data comprises a DICOM format and/or an ISMRMRD format.

The provided further measurement information comprises a data format that is dependent upon a data type of the provided further measurement information. If the further measurement information comprises physiological data, then the further measurement information may comprise e.g. an ISMRMRD format or further public community standard data formats and/or also proprietary data formats. If the further measurement information comprises further patient data, such as patient age and/or height of the patient and/or weight of the patient and so on, then the further measurement information may e.g. comprise data indicated in units such as meters, KG, years, and so on.

The at least one analysis module AM comprises the analysis module AM selected together with the measurement program MP. For analysis of the provided magnetic resonance data and/or the provided reconstructed image data and/or the provided further measurement information and for determination of the at least one item of evaluation information the at least one analysis module AM has a rule-based algorithm and/or machine learning-based algorithm. A rule-based algorithm is based on defined rules, in accordance with which this algorithm performs the task assigned to it. To solve the task a result may be compared with at least one threshold and from this a statement may be derived, e.g. the evaluation information.

A machine learning-based algorithm may e.g. comprise a trained machine learning method and has been trained to detect certain characteristics and/or a certain pattern in the input data to be analyzed. In general, a trained machine learning process mimics cognitive functions that humans associate with other human thoughts. In an embodiment, the training based on training data allows the machine learning method to adapt to new conditions and to detect and extrapolate patterns.

In an embodiment, a machine learning-based algorithm has been trained to detect certain characteristics and/or a certain pattern in the input data to be analyzed in relation to the question to be clarified. For the training, training data sets are used whose input data e.g. magnetic resonance data, for example k-space data and/or magnetic resonance raw data, and/or reconstructed image data and/or further measurement information, has already been evaluated with regard to a defined clinical question. In an embodiment, training data sets from different training patients are available.

The analysis of the input data by means of an analysis module AM may take place continuously throughout the measurement step MSi or throughout measurement steps MS1, MS2, . . . , MSn, for which the analysis module AM was selected. Furthermore, the provision of the evaluation information may e.g. also take place continuously throughout the measurement step MSi or throughout measurement steps MS1, MS2, . . . , MSn, for which the analysis module AM was selected.

The at least one analysis module AM may comprise at least one quality analysis module and/or at least one clinical analysis module and/or at least one technical analysis module and/or at least one general analysis module.

The at least one quality analysis module is designed to identify certain characteristics in input data, the certain characteristics relating to certain or specific data quality problems. In an embodiment, the at least one quality analysis module QAM is designed to check and analyze the input data continuously with regard to the defined or specific data quality problem. The presence of a data quality problem may, for example, adversely affect the image quality in the reconstructed image data such that a diagnostic evaluation of the image data is no longer possible or there is a danger of misdiagnosis due to the poor image quality. Furthermore, the at least one quality analysis module may monitor and analyze further measurement information, for example physiological data of the patient, during the execution of the measurement program MP. Such physiological data may, for example, be ECG data of the patient, acquired from the patient during the execution of the measurement program MP. Furthermore, such physiological data may also comprise data from the monitoring of the patient's breathing and/or further physiological data deemed useful by the person skilled in the art. The at least one quality analysis module may, for example, determine a probability of a problem and/or a critical state and/or an abnormality occurring in the image data, if no countermeasures are taken. The result of the analysis of the at least one quality analysis module comprises the at least one item of evaluation information. At the same time, the evaluation information may also comprise a measure of the probability of occurrence of a problem and/or an abnormality.

The at least one clinical analysis module may be designed to analyze the input data, e.g. the reconstructed image data for abnormalities. Such abnormalities may e.g. comprise a deviation from a norm. At the same time, the abnormality information may comprise neutral information, indicating for example an anomaly and an abnormality during the execution of at least one measurement step of the selected measurement program. Such abnormalities may furthermore also point to an illness, for example a suspected hemorrhage and/or a suspected heart attack. Based on the input data of the analysis module AM, for instance the reconstructed image data, a probability may be determined for the presence of an abnormality in the reconstructed image data of the at least one technical analysis module. If the determined probability exceeds a limit, this is evaluated and/or detected by the at least one clinical analysis module AM as an abnormality and/or anomaly, which is reflected in the evaluation information of the at least one clinical analysis module. At the same time, no diagnosis of the input data, e.g. the reconstructed image data, takes place by means of the clinical analysis module, it is merely an aid to a diagnosis by a doctor. In an embodiment, the analysis modules and the provided evaluation information and/or the provided assistance information should provide a doctor making a diagnosis with maximum support.

By means of the at least one quality analysis module and/or the at least one clinical analysis module a defined clinical question and/or a defined quality-related question and/or a patient-related question is analyzed as a function of the selected measurement program.

The at least one technical analysis module may be designed to analyze technical and/or device-related information, available to the measurement program MP. At the same time, by means of the at least one technical analysis module a defined technical question is analyzed as a function of the selected measurement program MP. For example, the at least one technical analysis module is designed for analysis of coil data of local high frequency coils. The local high frequency coils are positioned for acquisition of magnetic resonance data around the area to be examined of the patient. For different body areas, there are also different local high frequency coils available, for example, a head high frequency coil for head examination or a knee high frequency coil for knee examination. Based on the coil data for example the at least one technical analysis module may detect if for the selected measurement program MP the high frequency coil needed for this is connected with a scanner unit of a magnetic resonance device 10. Furthermore, the technical analysis module may also be designed for detection and/or early detection of hardware defects. For example, the at least one technical analysis module designed to analyze current operating parameters of local high frequency coils and to infer from this a status, e.g. a remaining service life, of the local high frequency coils or of individual components of the local high frequency coils.

The at least one general analysis module may be designed to monitor and to analyze general processes associated with the execution of the measurement program MP. At the same time, the at least one general analysis module may comprise a technical detection algorithm. Such a technical detection algorithm may, for example, be designed to detect and/or analyze the triggering of an image pre-processing. Alternatively or in addition, the at least one general analysis module may also comprise a setting-dependent detection algorithm. Such a setting-dependent detection algorithm may, for example, compare settings of the selected measurement program MP with existing software configurations and/or software licenses and/or with hardware configurations and in the event of deviations generate a warning that for execution of the measurement program MP not all software-relevant and/or scanner-relevant criteria have been met and therefore restrictions and/or problems may be expected during the execution of the measurement program MP. Alternatively or in addition, the at least one general analysis module may also be designed for analysis of general processes of the measurement program MP, such as an analysis for reporting.

The evaluation information determined by the at least one analysis module AM is also provided in this fourth method step 103. The provision of the at least one item of evaluation information takes place by means of the transaction manager TM.

In a further fifth method step 104 generation of assistance information takes place. The assistance information is generated as a function of the at least one item of evaluation information by means of the at least one analysis module AM. When multiple analysis modules AM1, AM2, . . . , AMn are used in the execution of a measurement step MSi each of the analysis modules AM1, AM2, . . . , AMn may generate assistance information. Furthermore, it may also be the case that when multiple analysis modules AM1, AM2, . . . , AMn are used in the execution of a measurement step MSi only one of the analysis modules AMi generates assistance information, e.g. if the analysis module AMi generating assistance information accesses the evaluation information of the further analysis modules AM1, AM2, . . . , AMn as input data.

The assistance information comprises problem information and/or abnormality information. The problem information is intended to indicate to a user a possible problem, associated with the execution of the measurement program MP and/or the magnetic resonance examination. The abnormality information may comprise information describing an abnormality of the patient, and hence a state deviating from a norm. Such abnormality information does not necessarily represent a quality problem in the acquired data. Rather, the abnormality information may also comprise neutral information indicating, for example, an anomaly and an abnormality during the execution of at least one measurement step of the selected measurement program.

Furthermore, the assistance information also comprises status information reflecting a status of the at least one analysis module AM. The status information comprises information on what type the analysis module AM is, for example if a quality analysis module and/or a technical analysis module and/or a clinical analysis module was selected for the assessment and/or analysis of the measurement step MSi. Furthermore, the status information may show the user if an analysis by means of the analysis module AM is currently being carried out. Furthermore, the status information may show the user if the analysis by means of the analysis module AM has been successfully completed and a result is available. Furthermore, the status information may display to the user if the analysis by means of the analysis module AM could not be carried out.

The at least one analysis module AM generates output data that is classified. The output data of the at least one analysis module comprises the evaluation information and/or the status information of the at least one analysis module AM. At the same time, different data classes are available for a classification. For classification of the output data of the analysis modules AM may comprise e.g. a traffic light classifier, a binary classifier, a multi-class classifier and a multi-class multi-label classifier are available. Categorization of the output data in an available classification comprises a semantic test of the question to be clarified of the analysis module AM concerned.

The generated assistance information is also provided in this fifth method step 104. The provision of the assistance information takes place by means of the transaction manager TM.

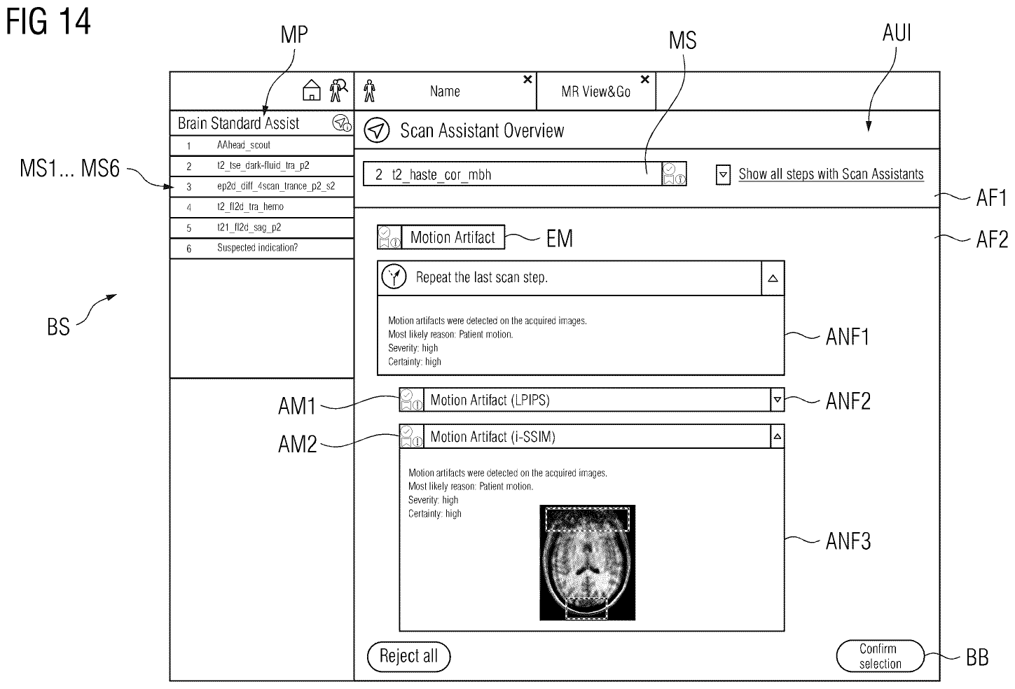

In a further sixth method step 105 output of the assistance information takes place. The output of the assistance information takes place an assistance user interface AUI, which is output via the user interface BS to a user. In an embodiment, at the user interface BS a visual output takes place by means of a display unit, e.g. a display and/or a monitor, to the user. At the same time, the output of the assistance information to the user takes place via a user interface BO for controlling and/or monitoring of the measurement program MP, the assistance user interface AUI being integrated into the user interface BO (see also FIG. 14).

The output of the status information of the analysis module AM is displayed by corresponding symbols, arranged at a defined position on the user interface BO. Examples of output symbols of the status information are shown in FIG. 12.

In the present exemplary embodiment, the problem information and/or the abnormality information is displayed at the user interface BO only upon active retrieval by the user. At the same time, by clicking on a symbol and/or button provided for that purpose the display of the problem information and/or the abnormality information may take place on the user interface BO. By displaying the status information the user is already informed that problem information and/or an abnormality information is present.

The provision of output data and the provision of the assistance information of the at least one analysis module AM and also the output of the assistance information may e.g. take place continuously throughout the entire measurement step MSi or measurement steps MS1, MS2, . . . , MSn, for which the analysis module AM was selected, so that a user experiences constant support.

FIG. 2 shows a first example of the assistance system AM. The assistance system AM is designed for execution of the method for supporting and/or assisting a user when executing a measurement program MP during magnetic resonance data acquisition, as described in FIG. 1. The assistance system AS comprises the transaction manager TM, the configuration interface CUI and at least one analysis module AM1, . . . , AMn. At the same time, in the present exemplary embodiment the assistance system AS may comprise 1 to n analysis modules AM1, . . . , AMn.

The measurement program MP comprises multiple measurement steps MS1, MS2, . . . . MSn. Information on the analysis modules AM1, . . . , AMn selected for the individual measurement steps MS1, MS2, . . . . MSn is provided to the transaction manager TM by means of the configuration user interface CUI. During execution of the first measurement step MS1 magnetic resonance data and/or further measurement information is continuously provided to the analysis modules AM1, . . . , AMn via the transaction manager TM. Each individual analysis module AM1, . . . , AMn analyzes and/or clarifies a defined question concerning the first measurement step MS1 and determines evaluation information and assistance information in the process. The assistance information is provided to the assistance user interface AUI by means of the transaction manager TM for output on the user interface BS. The assistance information is output on the user interface BS.

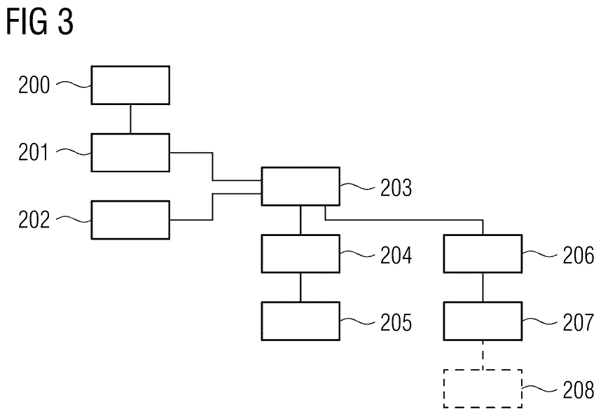

FIG. 3 shows an alternative exemplary embodiment of the method for supporting and/or assisting a user by means of an assistance system AS when executing a measurement program MP for magnetic resonance data acquisition. Components, features, and functions that stay the same are essentially identified by the same reference signs. The following description is essentially restricted to the differences from the exemplary embodiment in FIG. 1, with reference being made to the description of the exemplary embodiment in FIG. 1 for components, features, and functions that stay the same.

In a first method step 200, selection of a measurement program MP takes place for an acquisition of magnetic resonance data with a defined diagnostic question. Together with the measurement program MP, the user may also select support by means of the assistance system AS. At the same time, by selecting the measurement program MP, a selection of analysis modules AM already coordinated and/or assigned with/to the clinical and/or diagnostic question of the measurement program MP and/or a combination of analysis modules AM and recommendation modules EM coordinated with the clinical and/or diagnostic question of the measurement program MP may be offered to the user for selection. The measurement program MP comprises multiple measurement steps MS1, MS2, . . . , MSn, with for at least one measurement step MSi, support by means of the assistance system AS and consequently at least one analysis module AM and/or at least one recommendation module EM being selectable. Furthermore, for each of the multiple measurement steps MS1, MS2, . . . , MSn of the measurement program MP, support by means of the assistance system AS and consequently at least one analysis module AM and/or at least one recommendation module EM may also be selectable. The selection takes place by means of the configuration interface CUI on the user interface BS.

Apart from selection of the individual analysis modules AMi and/or recommendation modules EMi for the individual measurement step MSi, these may also be adapted to the respective measurement step MSi by means of the configuration user interface CUI. By means of the configuration user interface CUI the user may also select to what extent they wish to use support by means of the assistance system AS, for example automatic support, with which recommendations are automatically implemented by the assistance system AS, or semi-automatic support, with which the recommendation is only implemented following approval by the user, or simply output of information or recommendations.

Furthermore, with this first method step 200 it may also be the case that upon selection of an analysis module AM for at least one measurement step MSi of the measurement program MP recommendation modules EMi are already suggested for selection. The suggested recommendation modules EMi may e.g. be coordinated with the selected analysis module AM and may provide a suggestion for improving a problem and/or critical state and/or abnormality by means of the selected analysis module AM. The recommendation modules EMi are automatically suggested to the user by the assistance system AS, e.g. by means of the configuration interface CUI, upon selection of the analysis module AM.

Furthermore, with this first method step 200 it may also be the case that upon selection of a recommendation module EM for at least one measurement step MSi of the measurement program MP, at least one analysis module AM is linked with the recommendation module EM. Upon selection of the at least one recommendation module EM the at least one analysis module AM is automatically selected as well. For example, the selected recommendation module EM requires a data input provided by the analysis module AM linked with the recommendation module EM. The selection of analysis module AMi may e.g. take place automatically by means of the assistance system AS, e.g. by means of the configuration interface CUI.

At the same time, different analysis modules AMi and different recommendation modules EMi for different measurement steps MS1, MS2, . . . , MSn of the measurement program MP may be available and/or offered to the user for selection. The selection of the measurement program MP and also the selection of the at least one analysis module AM and/or recommendation module EM takes place by a user by means of the configuration interface CUI at the user interface BS.

In a second method step 201 execution of the selected measurement program MP takes place, the execution of the measurement program MP comprising acquisition of magnetic resonance data. Apart from the acquisition of magnetic resonance data image data is also reconstructed from the magnetic resonance data. The magnetic resonance data and the reconstructed image data are provided by the transaction manager TM. This second method step 201 is designed similarly to the method step 101 of the description in FIG. 1, to which reference is hereby made.

In a third method step 202 acquisition of further measurement information takes place, the further measurement information being acquired before execution of the selected measurement program MP or during execution of the selected measurement program MP. This third method step 202 is designed similarly to the method step 102 of the description in FIG. 1, to which reference is hereby made.

In a fourth method step 203 determination of at least one item of evaluation information takes place by means of at least one analysis module AM of the assistance system AS as a function of the provided magnetic resonance data and/or the provided image data and/or the provided further measurement information. This fourth method step 203 is designed similarly to the method step 103 of the description in FIG. 1, to which reference is hereby made.

In a fifth method step 204 generation of assistance information takes place, the assistance information being generated as a function of the at least one item of evaluation information by means of the at least one analysis module AM. This fifth method step 204 is designed similarly to the method step 104 of the description in FIG. 1, to which reference is hereby made.

In a sixth method step 205 output of the assistance information takes place. This sixth method step 205 is designed similarly to the method step 105 of the description in FIG. 1, to which reference is hereby made.

Simultaneously with the fifth and sixth method steps 204, 205 in a seventh method step 206 determination of at least one suggestion takes place for at least one measurement step MSi of the measurement program MP by means of the at least one recommendation module EM. At the same time, the determination of the at least one suggestion takes place based on at least one provided item of evaluation information of the at least one analysis module AM. For determination of the at least one suggestion the at least one recommendation module EM relies on suggestions adapted to a certain problem and/or a certain diagnostic question. At the same time, the at least one recommendation module EM may comprise multiple suggestions adapted to the certain problem and/or to the certain diagnostic question, each of which may contribute to correcting and/or solving and/or avoiding the problem and/or to clarifying an abnormality. These multiple suggestions may e.g. be stored in a database. It may also be that the provided evaluation information also holds information that favors a selection of a suggestion from the multiple suggestions by the at least one recommendation module EM.

Moreover, in this seventh method step 207 the recommendation module EM generates status information, comprising output data of the recommendation module EM. The output data of the recommendation module EM, e.g. the status information of the recommendation module, is classified to make different output data classes available. For a classification of the output data of the analysis modules, a traffic light classifier, a binary classifier, a multi-class classifier, and a multi-class multi-label classifier are available (see FIG. 12).

Furthermore, in this seventh method step 206 of the at least one recommendation module EM extended assistance information is determined, the extended assistance information comprising the at least one determined suggestion and/or the status information of the recommendation module AM. The extended assistance information is also provided for output to the user. The provision takes place by means of the transaction manager TM at the assistance user interface AUI, the assistance user interface AUI making available the extended assistance information at the user interface BS for output to the user.

In an eighth method step 207 output takes place of the extended assistance information determined by the at least one recommendation module EM. The extended assistance information comprises the suggestion and the status information. The extended assistance information is notified to the user via the assistance user interface AUI at the user interface BS. At the same time, the extended assistance information is output to the user via user interface BO for controlling and/or monitoring the measurement program MP.

Furthermore, the assistance system AS in a further, optional method step 208, may be designed for execution of the at least one suggestion for the at least one measurement step MSi. At the same time, the assistance system AS may be designed for execution of the at least one suggestion for the at least one measurement step MSi in a semi-automatic execution mode or in a fully-automatic execution mode. In an embodiment, in the first method step 200 a selection by the user may already have taken place of which execution mode they wish to have for the execution of the individual measurement steps MS1, MS2, . . . , MSn. At the same time, the user may also select different execution modes for different . . . , measurement steps MS1, MS2, . . . , MSn of the measurement program MP. The selection takes place via the user interface BS.

In the semi-automatic execution mode the user is again requested to make a confirmation entry prior to execution of the suggestion. For this purpose the assistance system AS generates a corresponding request for the confirmation entry and outputs it to the user via the user interface BO. As soon as the confirmation entry is present, the suggestion is then executed automatically by the assistance system AS. If no confirmation entry is made by the user, the actual user must carry out the individual steps or dispense with them. In the automatic execution mode the assistance system AS automatically executes the suggestion and the user is merely informed of the suggestion and its execution.

Furthermore, in this further, optional method step 208 it may also be provided that the user is requested following execution of the suggestion to enter feedback information, comprising a statement on a possible success of the suggestion executed. For this purpose the assistance system AS generates a corresponding request for the feedback information entry and outputs it via the user interface BO to the user.

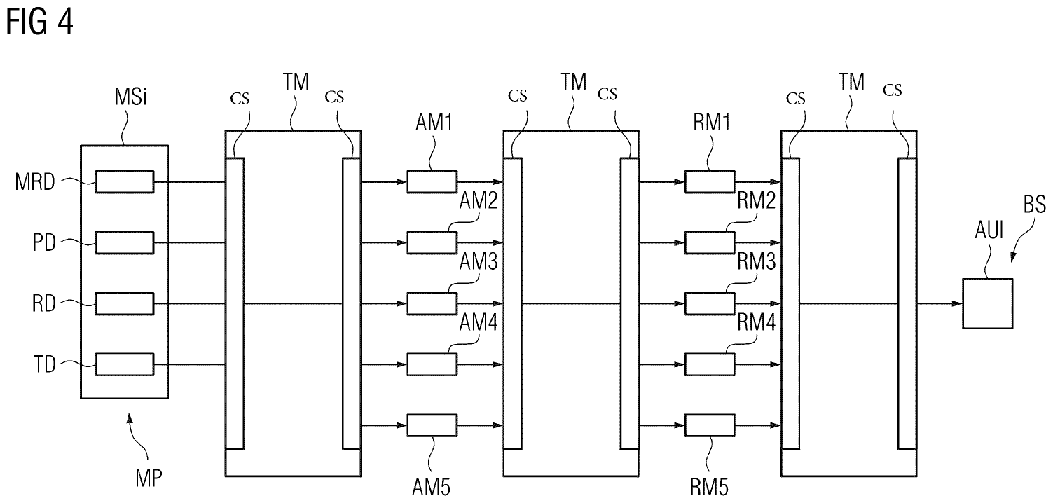

FIG. 4 shows an alternative exemplary embodiment of the assistance system AM. Components, features and functions that stay the same are essentially identified by the same reference signs. The following description is essentially restricted to the differences from the exemplary embodiment in FIG. 2, with reference being made to the description of the exemplary embodiment in FIG. 2 for components, features and functions that stay the same.

The assistance system AS in FIG. 4 comprises, apart from the transaction manager TM and analysis modules AM1, AM2, AM3, AM4, AM5, also recommendation modules EM1, EM2, EM3, EM4, EM5. Furthermore, the assistance system AS comprises the configuration user interface CUI, not shown in more detail in FIG. 4, reference being made to the description of FIG. 2 with regard to the functioning of the configuration user interface CUI. For execution of the inventive method of the magnetic resonance device the assistance system AS is provided with input data for analysis by means of the analysis modules AM1, AM2, AM3, AM4, AM5. The provision of the input data takes place by means of the transaction manager TM. Magnetic resonance data MRD, e.g. raw data and/or k-space data, and/or image data reconstructed from the magnetic resonance data is available as input data. Moreover, physiological data PD of the patient, such as e.g. ECG data and/or respiration data, is also available as input data. Moreover, further patient data RD, that has already been acquired during registration of the patient is available as input data. Moreover, hardware parameters TD, for example coil parameters and/or temperature parameters and so on, are also available as input data.

Five analysis modules AM1, AM2, AM3, AM4, AM5 are available for the analysis of the input data during the execution of the measurement program MP in the present exemplary embodiment. At the same time, the two analysis modules AM1 and AM2 are designed for analysis of the magnetic resonance data MRD and the reconstructed image data. Analysis module AM3 is designed for analysis of the physiological data PD. The analysis module AM4 is designed for analysis of the further patient data RD. The analysis module AM5 is designed for analysis of the hardware-parameters TD. Each of the analysis modules AM1, AM2, AM3, AM4, AM5 analyzes the input data in relation to the clinical and/or diagnostic question of the measurement program MP. From the analyzed input data the individual analysis modules AM1, AM2, AM3, AM4, AM5 may detect critical states and/or problems and/or abnormalities, that currently arise and/or may arise during execution of the measurement program MP, e.g. the measurement step MSi currently being executed. This information is contained in the evaluation information, evaluation information being determined by each of the analysis modules AM1, AM2, AM3, AM4, AM5. The evaluation information is provided by the transaction manager TM as output data of the analysis modules AM1, AM2, AM3, AM4, AM5.

The output data of the analysis modules AM1, AM2, AM3, AM4, AM5 is provided by the transaction manager TM as input data for recommendation modules EM1, EM2, EM3, EM4, EM5. In the present exemplary embodiment five recommendation modules EM1, EM2, EM3, EM4, EM5 are available for determination of suggestions. At the same time, the different recommendation modules EM1, EM2, EM3, EM4, EM5 may comprise an identical suggestion, the suggestion being designed for correcting different problems and/or different critical states and/or for clarification of abnormalities, for example in the image data. If the individual analysis modules AM1, AM2, AM3, AM4, AM5 have determined a critical state, the respective recommendation modules EM1, EM2, EM3, EM4, EM5 determine a suggested solution. At the same time, some recommendation modules EM1, EM2, EM3, EM4, EM5 may also have multiple suggestions and/or suggested solutions for selection.

The recommendation module EM1 determines as a function of the evaluation information of the analysis module AM1 a suggestion, the suggestion comprising repeating the current measurement step MSi. If the evaluation information comprises no critical state and/or no abnormality, the suggestion and/or the recommendation of the recommendation module EM1 comprises a continuation of the current measurement program MP. As a function of the analysis modules AM1, AM3 and AM4, the recommendation module EM2 determines a suggestion, wherein repeating the current measurement step MSi, changing parameter settings for the current measurement step MSi and adding at least one further measurement step MS are available as possible suggestions. If the incoming evaluation information comprises no critical state and/or no abnormality, the suggestion and/or the recommendation of the recommendation module EM2 comprises a continuation of the current measurement program MP. The recommendation module EM3 determines as a function of the analysis modules AM2 a suggestion, readjustment of parameters being available as a possible suggestion. If the evaluation information comprises no critical state and/or no abnormality, the suggestion and/or the recommendation of the recommendation module EM3 comprises a continuation of the current measurement program MP. The recommendation module EM4 determines as a function of the analysis modules AM3 a suggestion, addition of at least one further measurement step and adaptation of an ECG trigger signal being available as possible suggestions.

If the evaluation information comprises no critical state and/or no abnormality, the suggestion and/or the recommendation of the recommendation module EM4 comprises a continuation of the current measurement program MP. The recommendation module EM5 determines as a function of the analysis modules AM5 a suggestion, calibration of a hardware unit and informing a technical service and informing the user being available as possible suggestions. If the evaluation information comprises no critical state and/or abnormality, the suggestion and/or the recommendation of the recommendation module EM5 comprises a continuation of the current measurement program MP.

The respective recommendation module EM1, EM2, EM3, EM4, EM5 determines the suggestion that best corresponds to the evaluation information or the multiple evaluation information. Furthermore, the recommendation modules EM1, EM2, EM3, EM4, EM5 may also determine a common suggestion or multiple common suggestions. The determined suggestion and/or the determined suggestions are provided to the assistance user interface AUI by means of the transaction manager TM and output to the user at the user interface BS.

A data transfer between the magnetic resonance device and/or the analysis modules AM1, AM2, AM3, AM4, AM5 and/or the recommendation modules EM1, EM2, EM3, EM4, EM5 and/or the user interface BS takes place by means of the transaction manager TM. The transaction manager TM has for this a communication interface CS for data exchange between the magnetic resonance device and/or the analysis modules AM1, AM2, AM3, AM4, AM5 and/or the recommendation modules EM1, EM2, EM3, EM4, EM5 and/or the user interface BS.

In FIGS. 5 to 11 different exemplary embodiments of the assistance system AS are shown, the different exemplary embodiments comprising a different quantity of analysis modules AM1, AM2, AM3 and/or recommendation modules EM1, EM2. Data exchange between the individual analysis modules AM1, AM2 and AM3, the individual recommendation modules EM1, EM2, and the further units takes place in each case by means of the transaction manager TM. Selection and/or configuration of the individual analysis modules AM1, AM2, AM3 and/or recommendation modules EM1, EM2 takes place by means of the configuration user interface CUI, not shown in more detail in FIGS. 5 to 11.

In FIG. 5 support by means of the assistance system AS is selected for a measurement step MSi. At the same time, two analysis modules AM1, AM2 and one recommendation module EM1 have been selected. The analysis module AM1 determines evaluation information that serves as input data of the second analysis module AM2. The evaluation information determined by the second analysis module AM2 serves as input data for the recommendation module EM1. By means of the selected analysis modules AM1, AM2 and of the recommendation module EM1 a step by step artifact detection and artifact classification may be performed.

In measurement step MSi magnetic resonance data is acquired and from this image data with at least one image is reconstructed. This image data is provided by means of the transaction manager TM to the first analysis module AM1. The first analysis module AM1 determines based on the provided image data, e.g. of the at least one image, if there is an image quality problem present in the image data and generates from this evaluation information. The evaluation information may for example take the value “0”, if no image quality problem has been detected. Alternatively, the evaluation information may also take the value “1”, if an image quality problem has been detected in the image data by the first analysis module AM1.

The evaluation information is provided by the transaction manager TM to the second analysis module AM2. If the evaluation information of the first analysis module AM1 comprises the value “0”, no further analysis by means of the second analysis module AM2 takes place. If the evaluation information of the first analysis module AM1 comprises the value “1”, by means of the second analysis module AM2 a further analysis of the image data is carried out. At the same time, by means of the second analysis module AM2 it is determined what type the image quality problems may be and/or what the cause of the image quality problems in the provided image data may be. For example, motion artifacts may be determined as the cause of 70% of the image quality problems and Gibbs ringing may be determined as the cause of 10% of the image quality problems. The result is made available by the second analysis module AM2 as evaluation information. Furthermore, the information, that image quality problems have been detected and/or were the cause of the image quality problems of the first analysis module AM1 and/or second analysis module AM2, is also provided as assistance information for output to the user. This assistance information is provided by the transaction manager TM to the assistance user interface AUI, assistance user interface AUI making the assistance information available at the user interface BS for output to the user.

The evaluation information of the second analysis module AM2 is provided by the transaction manager TM to the recommendation module EM1. The recommendation module EM1 then determines which further action is most likely to lead to a solution of the image quality problem. For example, the recommendation module EM1 may comprise a rule-based algorithm, which based on the evaluation information of the second analysis module AM2 determines a most probable solution. Based on this most probable solution the recommendation module EM1 generates a suggestion and/or a recommendation for the user. An example of a suggestion and/or a recommendation may be: “The last measurement step should be repeated.” This suggestion and/or this recommendation is provided by the recommendation module EM1 as extended assistance information.

The extended assistance information is then provided by the transaction manager TM to the assistance user interface AUI, with the assistance user interface AUI making the extended assistance information available to the user interface BS for output to the user. For example, the following information may be displayed to the user: “A motion artifact was detected. Do you wish to repeat the last measurement step?” The user now has the opportunity to manually accept or reject the recommendation at the user interface BS. If the recommendation is accepted it is automatically implemented by the assistance system AS. If it is rejected the measurement program MP is continued manually.

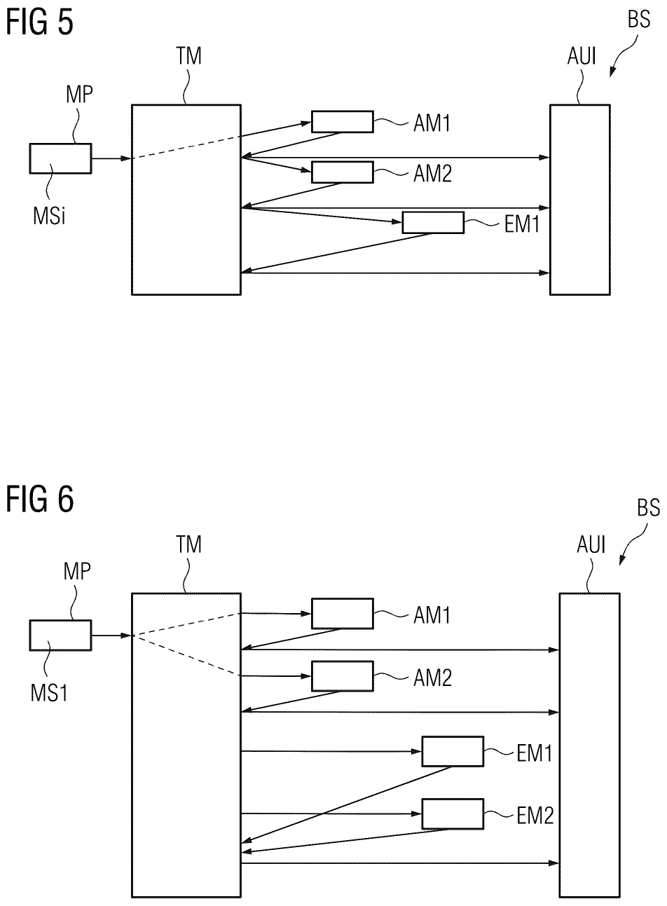

In FIG. 6 support is selected for a measurement step MS1 by means of the assistance system AS. At the same time, two analysis modules AM1, AM2 and two recommendation modules EM1, EM2 have been selected. The two analysis modules AM1, AM2 determine evaluation information, independently of one another, which serves as input data for the recommendation modules EM1, EM2.

In measurement step MS1 magnetic resonance data is acquired and from this image data with at least one image is reconstructed. This image data is provided by means of the transaction manager TM to the first analysis module AM1 and the second analysis module AM2. The first analysis module AM1 analyzes the provided image data with regard to motion artifacts. The second analysis module AM2 analyzes the provided image data with regard to noise behavior of the image data. The two analysis modules AM1, AM2 each determine evaluation information independently of one another and also make assistance information available. This assistance information is provided by the transaction manager TM to the assistance user interface AUI, the assistance user interface AUI making the assistance information available at the user interface BS for output to the user.

The evaluation information is provided by the transaction manager TM to the recommendation modules EM1, EM2. At the same time, the evaluation information of the first analysis module AM1 is provided by the transaction manager TM to the first recommendation module EM1. The first recommendation module EM1 determines with which measures the motion artifacts detected in the image data by the first analysis module AM1 could be reduced and/or prevented. For example, one measure may be to repeat the measurement step for detection of the image data. For this purpose the first recommendation module EM1 determines a corresponding suggestion and/or recommendation and provides this as extended assistance information for output to a user.

The evaluation information of the second analysis module AM2 is provided by the transaction manager TM to the second recommendation module EM2. The second recommendation module EM2 determines with what measures the image noise detected by the second analysis module AM2 may be suppressed and the signal-to-noise ratio improved. For example, one measure may be to set at least one average or also multiple averages. Depending on the type and/or intensity of the detected image noise this measure may be recommended only for the further measurement steps MSi or also for a repetition of the first measurement step MS1. For this purpose, the first recommendation module EM2 determines a corresponding suggestion and/or recommendation and provides this as extended assistance information for output to a user.

The extended assistance information is then provided by the transaction manager TM to the assistance user interface AUI, with the assistance user interface AUI making the extended assistance information available to the user interface BS for output to the user and displaying this to the user via the user interface BS. The user now has the opportunity to manually accept or reject the recommendation at the user interface BS. If the recommendation is accepted it is automatically implemented by the assistance system AS. If it is rejected the measurement program MP is continued manually.

The method from FIG. 6 may, for example, be applied during head imaging for parallel checking of image quality problems of various types.

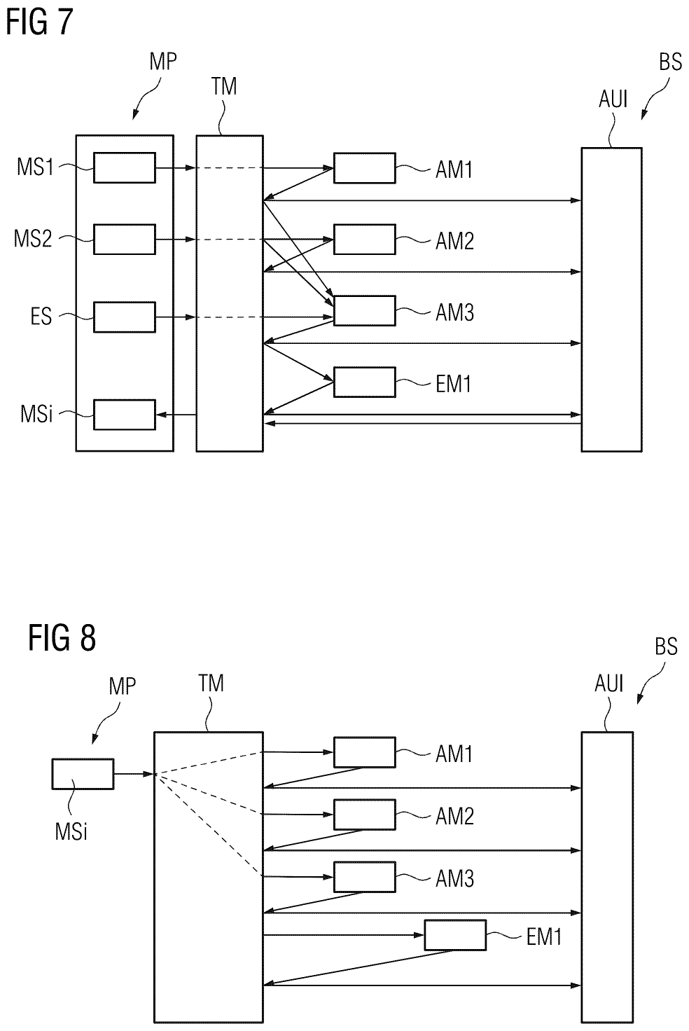

In FIG. 7 support for multiple measurement steps MS1, MS2 is selected by means of the assistance system AS. At the same time, for the first measurement step MS1 a first analysis module AM1, and for a second measurement step MS2 a second analysis module AM2 have been selected. A third step comprises a decision step ES, in which a decision is made on the further course of the measurement program MP. For this decision step ES a third analysis module AM3 has been selected. At the same time, it may also be the case that the third analysis module AM3 has only been selected by a user for the decision step ES, and the configuration user interface CUI has also automatically selected both analysis modules AM1, AM2 for the first and second measurement steps MS1, MS2. A recommendation module EM1 has also been selected for the decision step ES. At the same time, it may also be the case that only recommendation module EM1 has been selected by a user only for the decision step ES, and the configuration user interface CUI has also automatically selected the three analysis modules AM2, AM2, AM3 for the corresponding measurement steps MS1, MS2.

In decision step ES it is decided which further measurement steps MSi will be executed after the decision step ES. This decision is based on the results of the first two measurement steps MS1, MS2. At the same time, the user may select if they want to have only one recommendation displayed of which further measurement steps MSi would be useful for clarification of abnormalities or if a certain event occurs, but with the decision and above all selection of the further measurement steps MSi being left to the user. Alternatively, the user may also select automated support for this decision step ES, with which a measurement path is recommended to the user if a certain event occurs, the measurement path already having been prepared and configured by the assistance system and being ready for execution.

The method from FIG. 7 may, for example, be applied during head imaging. Here, parallel checking of the patient for abnormalities may be carried out, the detected or discovered abnormalities being the basis for a decision on the further course of the measurement program MP.

In the first measurement step MS1, magnetic resonance data is acquired and from this first image data with at least one image is reconstructed. This first image data is provided to the first analysis module AM1 by means of the transaction manager TM, the first analysis module AM1 analyzing the first image data with regard to a midline shift of the brain. The first analysis module AM1 determines evaluation information and also makes assistance information available, which by means of the transaction manager TM is made available to the assistance user interface AUI for output at the user interface BS. The assistance information may, for example, in the event of a detected midline shift comprise the information: “Midline shift of the brain detected”.

In the second measurement step MS2 magnetic resonance data is acquired and from this second image data with at least one image is reconstructed. This second image data is provided to the second analysis module AM2 by means of the transaction manager TM, the second analysis module AM2 analyzing the second image data with regard to hyperintensity of the white brain matter. The second analysis module AM2 determines evaluation information and also makes assistance information available, which by means of the transaction manager TM is made available to the assistance user interface AUI for output at the user interface BS. The assistance information may, for example, comprise the information: “Hyperintensity of the white brain matter detected”.

In decision step ES, the third analysis modules AM3 is notified by means of the transaction manager TM that a workflow adjustment and/or an adjustment of the measurement program should be made at this point in the measurement program MP. For this purpose the third analysis module AM3 analyzes both the first image data and the second image data, provided by means of the transaction manager TM. For example, the third analysis module AM3 may analyze the first and second image data with regard to a spatially correlated abnormality and from this determine the need for a perfusion measurement. The third analysis module AM3 determines evaluation information and also makes assistance information available, which by means of the transaction manager TM is made available to the assistance user interface AUI for output at user interface BS. The assistance information may, for example, comprise the information: “Perfusion measurement required”.

The evaluation information of the third analysis module AM3 is provided by the transaction manager TM to the recommendation module EM1. The recommendation module EM1 determines on the basis of the evaluation information of the third analysis module AM3 a recommendation and/or a suggestion and provides this as extended assistance information for a user. The extended assistance information is then provided by the transaction manager TM to the assistance user interface AUI, with the assistance user interface AUI making the extended assistance information available to the user interface BS for output to the user and displaying this to the user via the user interface BS.

If when selecting the measurement program MP for the decision step ES the user has selected only a display of a recommendation, it is now recommended to the user by display of the extended assistance information via the user interface BS, that they make a corresponding adjustment to the measurement program MP, e.g. to the further measurement steps MSi. In the present exemplary embodiment, the recommendation is a perfusion measurement as one of the next measurement steps MSi in the measurement program. The user may manually select the individual measurement steps MSi or also accept an automatic adjustment of the measurement steps MSi by the assistance system AS.

If when selecting the measurement program MP for the decision step ES the user has selected the automated support, they receive a suggestion for a measurement path for continuation of the measurement program MP as a recommendation and/or as extended assistance information via the user interface BS. The recommendation module EM1 determines a measurement path from multiple available measurement paths, that best leads to clarification of the abnormalities and/or the results detected and/or analyzed in the analysis modules AM1, AM2, AM3. In the present exemplary embodiment, the recommendation contains a measurement path with a perfusion protocol and/or a perfusion measurement. This measurement path is already preconfigured and ready for execution and only needs to be accepted by the user via the user interface BS. Furthermore, the user also has the opportunity to reject the suggestion via the user interface BS and continue with the measurement program MP manually.

In FIG. 8 support by means of the assistance system AS is selected for a measurement step MSi. At the same time, three analysis modules AM1, AM2, AM3 and one recommendation module EM1 have been selected. The analysis modules AM1, AM2, AM3 determine evaluation information independently of one another, which serves as input data for the recommendation module EM1. This exemplary embodiment is an example of multiple analysis modules AMi, which analyze different data, generated by the same measurement step MSi. An exemplary application for this is cardiac imaging quality assurance, in which three different analysis modules AM1, AM2, AM3 analyze three different input data, for example image data, ECG data and a respiration curve of the patient. Each analysis module AM1, AM2, AM2 may detect signal-specific problems. The results, e.g. evaluation information, of the analysis modules AM1, AM2, AM3 are consolidated in a subsequent recommendation module EM1, to determine a final recommendation to the user.

In measurement step MSi magnetic resonance data and further measurement information are acquired, and the acquired magnetic resonance data and/or image data reconstructed from this and the further measurement information are made available by means of the transaction manager TM to the individual analysis modules AM1, AM2, AM3 for analysis. The provided measurement information comprises ECG information and respiratory information.

The first analysis module AM1 analyzes the provided image data, comprising image data of the patient's heart, with regard to quality problems, e.g. motion artifacts in the image data. Evaluation information determined from this may, for example, take a value “0” or “1” and corresponding assistance information is generated. At the same time, the evaluation information “0” comprises, for example, the assistance information “no motion artifacts detected” and “1” the assistance information “motion artifacts detected”.

The second analysis module AM2 analyzes the ECG information. At the same time, the ECG information may comprise both ECG signals of the patient and also data on positioning of the ECG electrodes or also data on ECG electrode connectivity. The second analysis module AM2 may detect both an arrhythmia in the ECG signals and the positioning and connectivity of the electrodes. Evaluation information determined from this may, for example, take a value “0” or “1” and corresponding assistance information is generated. At the same time, the evaluation information “0” comprises, for example, assistance information “no arrhythmia detected” and/or “electrodes correctly positioned” and/or “electrodes correctly connected” and “1” the assistance information “arrhythmia detected” and/or “electrodes incorrectly positioned” and/or “electrodes incorrectly connected”.

The third analysis module AM3 analyzes the respiratory information with regard to patient compliance with specified breathing commands. Evaluation information determined from this may, for example, take a value “0” or “1” and corresponding assistance information is generated. At the same time, the evaluation information “0”, for example, comprises the assistance information “patient has correctly followed breathing instructions” and “1” the assistance information “patient has not followed breathing instructions”.

The individual assistance information is made available by means of the transaction manager TM to the assistance user interface AUI for output to user interface BS.

The recommendation module EM1 receives by means of the transaction manager TM the provided evaluation information of the individual analysis modules AM1, AM2, AM3. The recommendation module EM1 analyzes the results of the three analysis modules AM1, AM2, AM3, for whether overall a quality problem is present, and if a quality problem is present, what the most probable cause of the problem is and how this may best be mitigated. If, for example, by means of the second analysis modules AM2 an arrhythmia is detected, the recommendation module EM1 may determine the suggestion and/or the recommendation to repeat the measurement step MSi with a protocol that responds less sensitively to arrhythmia, such as a compressed sensing CINE protocol. This recommendation and/or the suggestion is or are provided as extended assistance information. The extended assistance information is then provided by the transaction manager TM to the assistance user interface AUI, wherein the assistance user interface AUI makes the extended assistance information available to the user interface BS for output to the user and displays this to the user via the user interface BS. An example of a recommendation text would be: “In the last measurement step, CINE-LAX′ motion was determined. The cause is probably an arrhythmia of the patient. Do you want to repeat the measurement with the more robust, CS CINE LAX′ protocol?” The user may then accept or reject the recommendation and continue manually.

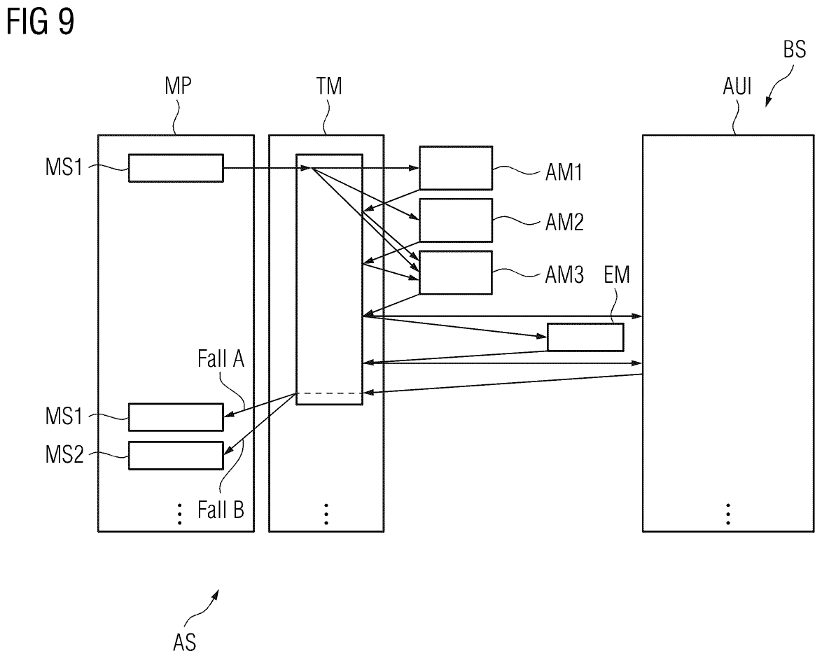

FIG. 9 shows the assistance system AS in a serial execution mode. For a first measurement step MS1 multiple analysis modules AM1, AM2, AM3 and one recommendation module EM1 are selected. Data exchange between the individual analysis modules AM1, AM2, AM3 and the recommendation module EM1 takes place by means of the transaction manager TM. In this execution mode the next measurement step MS2 is only started when the first measurement step MS1 and consequently also support by means of the assistance system is complete. In the present exemplary embodiment the first measurement step MS1 is therefore only completed when the suggestion recommended by the recommendation module EM1 is carried out or rejected by the user at the user interface BS.

In the first measurement step MS1 magnetic resonance data is acquired and from this image data with at least one image is reconstructed. This image data is provided by means of the transaction manager TM to the three analysis modules AM1, AM2, AM3. The first analysis module AM1 quantifies the degree of image blurring on a reconstructed image by means of an algorithm A. An example of evaluation information would be a scalar with the value “0.3” and which also provides assistance information. The second analysis module AM2 quantifies the degree of image blurring on a reconstructed image by means of an algorithm B. An example of evaluation information would be a scalar with the value “0.8” and which also provides assistance information.

The evaluation information of both analysis modules AM1, AM2 is provided by means of the transaction manager TM to the third analysis module AM3, which operates as a secondary analysis module AM3 and creates consolidated evaluation information. An example of consolidated evaluation information of the third analysis modules AM3 would be the value “0.5”, which the third analysis module AM3 determines on the basis of both items of evaluation information of both first analysis modules AM1 and AM2. As assistance information an average quality class is determined with the output “Image sharpness—Poor”. The individual assistance information is made available to the configuration user interface CUI by means of the transaction manager TM for output at the user interface BS.

The recommendation module EM1 receives by means of the transaction manager TM the provided evaluation information of the third analysis module AM3 and determines from this a suggestion and/or a recommendation for the user. At the same time, the recommendation module EM1 may comprise a rule-based algorithm. An example recommendation in the present exemplary embodiment would be “Check the image sharpness and consider increasing the image resolution. Increase image resolution by 20%?” This recommendation and/or this suggestion may be recommended for the subsequent measurement step MS2 or also for a repetition of the first measurement step MS2. This recommendation and/or this suggestion is or are provided by the recommendation module EM1 as extended assistance information and then provided by the transaction manager TM to the assistance user interface AUI, wherein the assistance user interface AUI makes the extended assistance information available to the user interface BS for output to the user and displays this via the user interface BS. The user may accept the recommendation (Case A), and the recommendation is correspondingly implemented by the assistance system AS. If the user rejects the recommendation (Case B) a manual continuation of the measurement program MP takes place.

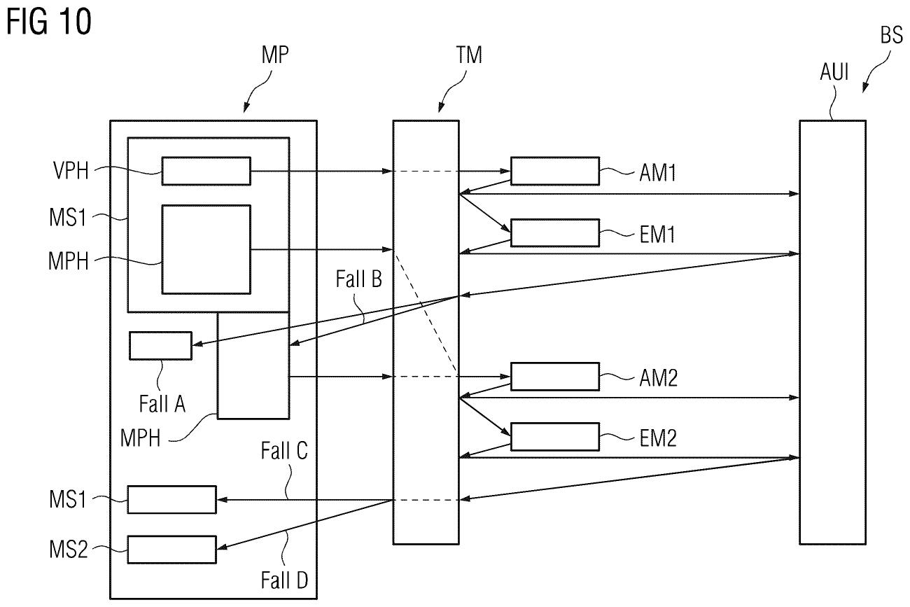

FIG. 10 shows the assistance system AS in a quasi-real time mode. Here, a first measurement step MS1 comprises a preparation phase VPH and a measurement phase MPH. An exemplary application of this configuration is the monitoring of the patient's physiological signals which may have a significant impact on the diagnostic quality of the acquired magnetic resonance data. An analysis of these physiological signals may take place in subsegments, while the MR measurement step MS1 continues uninterrupted. The preparation phase VPH is, for example, designed to determine the time for which the patient holds their breath. This data is forwarded to a first analysis module AM1 and analyzed there. While the first analysis module AM1 analyzes the respiration data, the measurement phase MPH of the first measurement step MS1 starts. A data exchange between the individual analysis modules AM1, AM2 and the recommendation modules EM1, EM2 takes place by means of the transaction manager TM.

During the preparation phase VPH, the first analysis module AM1 analyzes the patient's respiration curve to see if they are able to comply with the breathing commands (for example “breathe in-breathe out-breathe in and hold breath”). Evaluation information determined from this may, for example, take a value “0” or “1” and corresponding assistance information is generated. At the same time, evaluation information “0”, for example, comprises the assistance information “The patient successfully followed the preparation phase VPH” and “1” the assistance information “The patient has not successfully followed the preparation phase VPH”. The assistance information is made available by means of the transaction manager TM to the assistance user interface AUI for output at the user interface BS.

The first recommendation module EM1 determines based on the provided evaluation information of the first analysis module AM1 a recommendation and/or a suggestion. In the event that the patient is able to follow the breathing instructions, the suggestion and/or the recommendation would be to simply continue with the measurement program MP without making changes. In the event that the patient is unable to follow the breathing instructions, a possible recommendation of the recommendation module EM1 would be to discontinue the measurement step MS1 and check if the patient is able to hear and/or understand etc. and then repeat the measurement. The recommendation and/or the suggestion is provided as extended assistance information by means of the transaction manager TM to the assistance user interface AUI, wherein the assistance user interface AUI makes available the extended assistance information of the user interface BS for output to the user and displays this to the user via the user interface BS. At the same time, the recommendation may also be displayed step by step with the user being asked to make a confirmation entry or discontinuation entry at each step.

If the user rejects the recommendation (Case A) the measurement program MP is continued manually by the user (not described in detail here). If the user accepts the suggestion (Case B), an analysis by a second analysis module AM2 also takes place. The second analysis module AM2 analyzes the breath holding phase, e.g. the respiration curve and the breath holding time, of the patient during the measurement phase MPH. If the patient has successfully completed the breath holding phase, the evaluation information “O” is determined and provided. If on the other hand the patient has not successfully completed the breath holding phase, the evaluation information “1” is determined and provided. Furthermore, the evaluation information may also comprise information on how long the patient manages to hold their breath.

A second recommendation module EM2 determines, based on the evaluation information of the analysis module AM2, a recommendation and/or a suggestion. In the event of the patient being able to follow the breathing instructions, the second recommendation module EM2 would not determine a new suggestion and/or recommendation. In the event of the patient not being able to follow breathing instructions, a possible recommendation of the second recommendation module EM2 would be to reduce the time for which the patient holds their breath, for example by 4 s. Then measurement step MS1 should be restarted. This recommendation and/or this suggestion is provided by the recommendation module EM2 as extended assistance information and then provided by the second transaction manager TM to the assistance user interface AUI, wherein the assistance user interface AUI makes the extended assistance information available to the user interface BS for output to the user and displays this via the user interface BS. The user may accept the recommendation (Case C) and repeat measurement step MS1 or reject it (Case D) and continue with measurement step MS2.

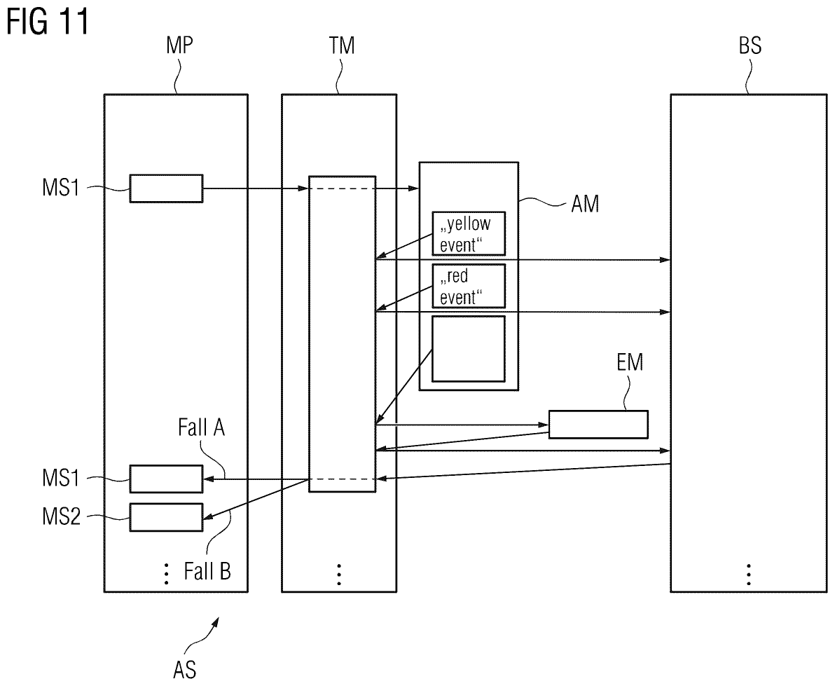

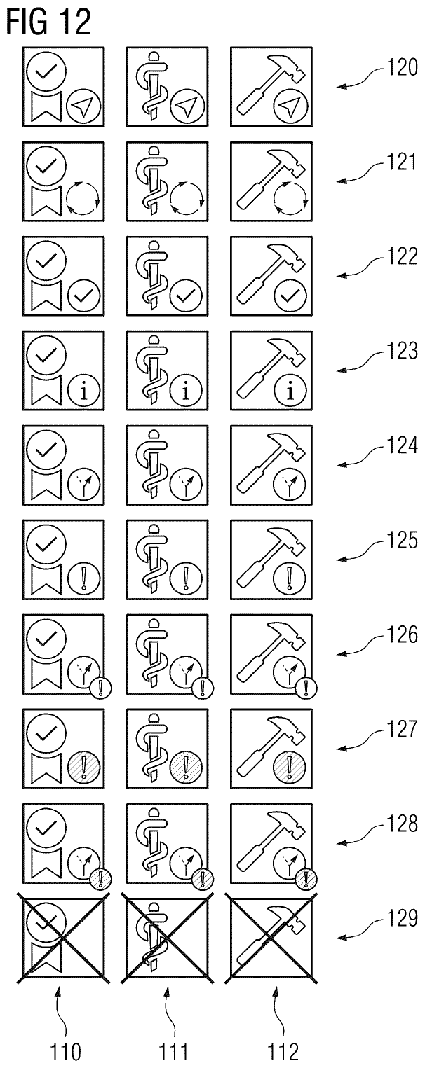

FIG. 11 shows the assistance system AS in a real time mode, in which an analysis of measurement data is being carried out while the measurement is running. For certain quality analyses it may be an advantage to make feedback available as quickly as possible to the user. The analysis module AM1 constantly analyzes the incoming data and continuously determines current evaluation information and assistance information, in order to continuously provide the user with feedback on the current measurement. Data exchange between the analysis module AM1 and the recommendation module EM1 takes place by means of the transaction manager TM.

The analysis module AM1 carries out an analysis of the physiological data in real time, e.g. immediately, while the data is being generated and the measurement, e.g. the first measurement step MS1, continues to run. Here, for example, motion artifacts in the image data may be detected and/or recognized in real time. At the same time, over the course of the measurement different and possibly transient criticality levels may be reached. At the same time, the analysis module AM1 continuously determines for example the measurement quality and consequently evaluation information and assistance information. For example, the evaluation information may be “Good” immediately after the start of the measurement and correlated with a green display symbol as assistance information. As soon as the analysis module AM1 acquires a change, for example motion, changed evaluation information is generated, for example “Uncertain” with this being correlated with a yellow display symbol as assistance information. The change in the measurement quality may also lead to changed evaluation information with the value “Poor” and this being correlated with a red display symbol as assistance information. As soon as the measurement quality improves again, the corresponding evaluation information is also changed to “Uncertain” or “Good” and the assistance information also comprises a yellow display symbol or a green display symbol. The evaluation information is continuously made available in real time by means of the transaction manager TM to the recommendation module EM1. The assistance information is made available by means of the transaction manager TM to the assistance user interface AUI for output at the user interface BS.

At the end of the analysis the analysis module AM1 outputs a final result. At the same time, the extent to which the measurement quality is subject to fluctuations may be decisive. If for most of the time the measurement quality is “Good” and only briefly “Uncertain” or “Poor”, the final result may comprise evaluation information with the value “0” and “Good”. If, on the other hand, for most of the time the measurement quality is “Poor” and remains at this level, the final result from the AM1 analysis module comprises evaluation information with the value “1” and “Poor”.

The recommendation module EM1 determines based on the evaluation information of the analysis module AM1 a recommendation to the user. If, for example, the evaluation information has the value “0”, only a green display symbol would be displayed for the assistance information, since there is no need for corrective action. If, on the other hand, the evaluation information has the value “1”, discontinuation and a repeat of measurement step MS1 is suggested by the recommendation module. Corresponding extended assistance information could be: “The measurement should be discontinued due to motion artifacts. Do you want to restart the measurement?”. This recommendation and/or this suggestion is provided by the recommendation module EM as extended assistance information and then provided by the transaction manager TM to the assistance user interface AUI, wherein the assistance user interface AUI makes the extended assistance information available to the user interface BS for output to the user and displays this via the user interface BS.

The user may accept the recommendation of the recommendation module EM1 (Case A) and the first measurement step MS1 is repeated or reject the suggestion (Case B) and manually continue with the second measurement step MS2.

FIG. 12 shows the classification of the output data of the analysis modules AM and the receive modules EM in tabular form. The first column 110 shows the symbols available for quality modules, the second column 111 the symbols available for clinical modules and the third column 112 the symbols available for technical modules.

A first line 120 indicates that a module, e.g. an analysis module AM and/or a recommendation module EM, has been selected. In a second line 121 the symbols are shown when the corresponding module, e.g. an analysis module AM and/or a recommendation module EM, is currently carrying out an analysis. The symbols in lines 120 and 121 are available for output data of all classes.

In lines 122, 123 and 124 the symbols are shown when the corresponding module, e.g. an analysis module AM and/or a recommendation module EM, has successfully completed the analysis and no problem and/or no abnormality has been detected. Line 122 is for analysis modules AM and/or recommendation modules EM, which provide output data of the traffic light classifier class. For this class no problem and/or no critical state and/or no abnormality exists and no further action by the user is required. Line 123 is for an analysis module AM and line 124 for a recommendation module EM, which provide output data of the multi-class classifier and multi-class multi-label classifier classes. The analysis has been successfully completed and with sufficient certainty and no action is required, but a recommendation and/or a suggestion may be present.

In lines 125 and 126 the symbols are shown when the corresponding module, e.g. an analysis module AM and/or a recommendation module EM, has successfully completed the analysis, but the result is uncertain. Line 125 is for an analysis module AM both of the traffic light classifier class and also the multi-class classifier and multi-class multi-label classifier classes. Action by the user, for example execution of a suggestion, is recommended by the analysis modules AM. Line 126 is for a recommendation module EM both of the traffic light classifier class and also the multi-class classifier and multi-class multi-label classifier classes. The recommendation modules EM provide a suggestion for the user.

In lines 127 and 128 the symbols are shown, when the corresponding module, e.g. an analysis module AM and/or a recommendation module EM, has successfully completed the analysis and a problem and/or critical state and/or an abnormality has been detected. Line 127 is for analysis modules AM of the traffic light classifier class. Action by the user, for example execution of a suggestion, is recommended by the analysis modules AM. Line 128 is for a recommendation module EM of the traffic light classifier class. The recommendation module EM provides a suggestion for the user.

In lines 129 the symbols are shown when the corresponding module, e.g. an analysis module AM and/or a recommendation module EM, could not complete the analysis. There is therefore no result. The symbols are intended for modules, e.g. analysis modules AM and/or recommendation modules EM, both of the traffic light classifier class and the multi-class classifier and multi-class multi-label classifier classes.

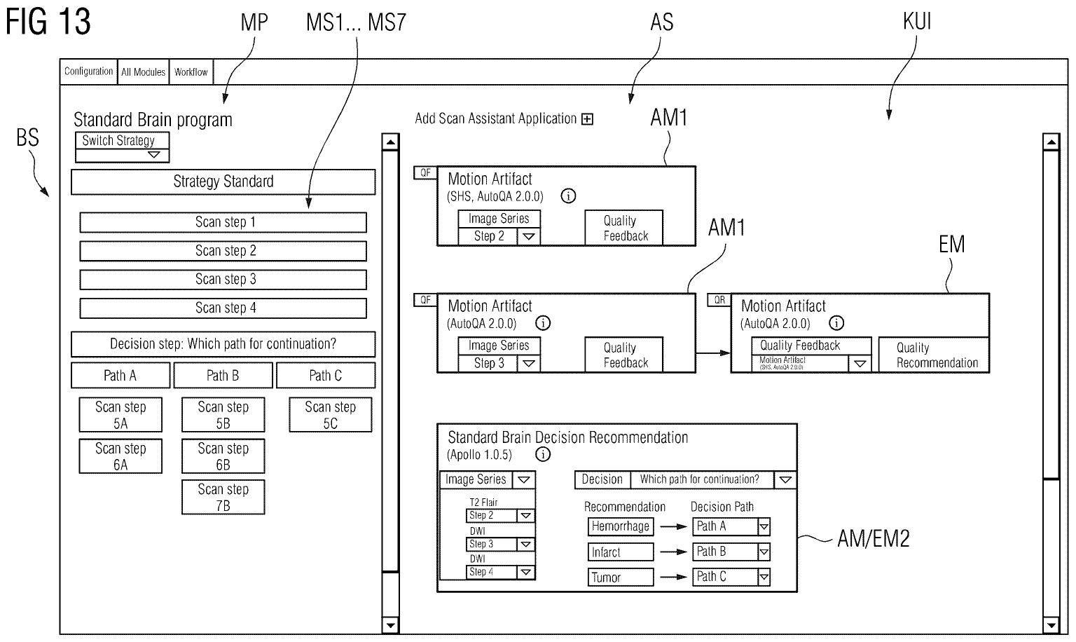

FIGS. 13 and 14 each show a user interface BO for communication by the assistance system AS with a user.

FIG. 13 shows the user interface BO in a selection mode of the configuration user interface CUI. In this selection mode the user may, apart from selection of a measurement program MP for individual measurement steps MS1 to MS7, select support by means of the assistance system AS. At the same time, the user may select a type of analysis module AM. Furthermore, the user may also consequently select a linked recommendation module EM. In the present exemplary embodiment the measurement program MP comprises a head examination. The individual measurement steps MS1 to MS7 of the measurement program are shown one after another continuously on the left side of the user interface BO. The selection of the individual modules of the assistance system AS takes place via the right half of the user interface BO. At the same time, for measurement step MS2 a first analysis module AM1 for detection of motion artifacts is selected. For measurement step 4 again the first analysis module AM1 for detection of motion artifacts is also selected, but this time together with a first recommendation module EM, designed to determine a suggestion.

Furthermore, decision support may also be selected by the user by means of the assistance system AS. At the same time, it may be defined which measurement path is to be selected if a certain event occurs. In the present exemplary embodiment, the event comprises a suspicion of a certain illness of the patient. If by means of the assistance system AS decision support is selected, a user may define which measurement path is to be selected if a certain event occurs. The assistance system AS supports the user in detecting and/or determining the occurrence of the certain event. In the present exemplary embodiment, a second analysis module AM2 and a second recommendation module EM2 are available, the user being able to select which measurement path is output as a suggestion, when a certain event occurs. If the second analysis module AM2 determines and/or identifies a suspicion of a cerebral hemorrhage, measurement path 1 is selected as a suggestion by a second recommendation module EM2 and displayed to the user. If, on the other hand, the second analysis module AM2 determines and/or identifies a suspicion of an infarction, measurement path 2 is selected as a suggestion by the second recommendation module EM2 and displayed to the user. If, furthermore, the second analysis module AM2 determines and/or identifies a suspicion of a tumor, measurement path 3 is selected as a suggestion by the second recommendation module EM2 and displayed to the user.

FIG. 14 shows the user interface BO in a recommendation mode of the assistance user interface AUI for a measurement program MP. The measurement program comprises multiple measurement steps MS1 to MS6, the individual measurement steps MS1 to MS6 of the measurement program being shown one after another continuously on the left side of the user interface BO. Output of assistance information and/or a suggestion and/or status information of the selected recommendation module EM takes place on the right half of the user interface BO.

In a first display field AF1 the measurement step MS is displayed for which support by means of the assistance system AS is present. Below this is a second display field AF2, which initially displays a status of the selected recommendation modules EM by a symbol. Next to this is a name of the selected recommendation module EM. Below this a first display window ANF1 is arranged, having status information of the recommendation module EM and also briefly displaying the suggestion of the recommendation module EM. In an enlargement of the first display window ANF1, which the user must manually click, the user may receive further information of the recommendation module EM. Below this two further display windows ANF2, ANF3 are shown indented, which identify an analysis module AM1, AM2 respectively and also comprise status information of the respective analysis module AM1, AM2. These two display windows ANF2, ANF3 may also be enlarged by manually clicking them, so that further assistance information, e.g. problem information and/or abnormality information determined by the respective analysis module AM1, AM2 is visible to the user. In the bottom right of the display field AF2 is a confirmation button BB, which the user may press and/or click, if they wish the assistance system AS to automatically execute the suggestion suggested by the recommendation module EM.

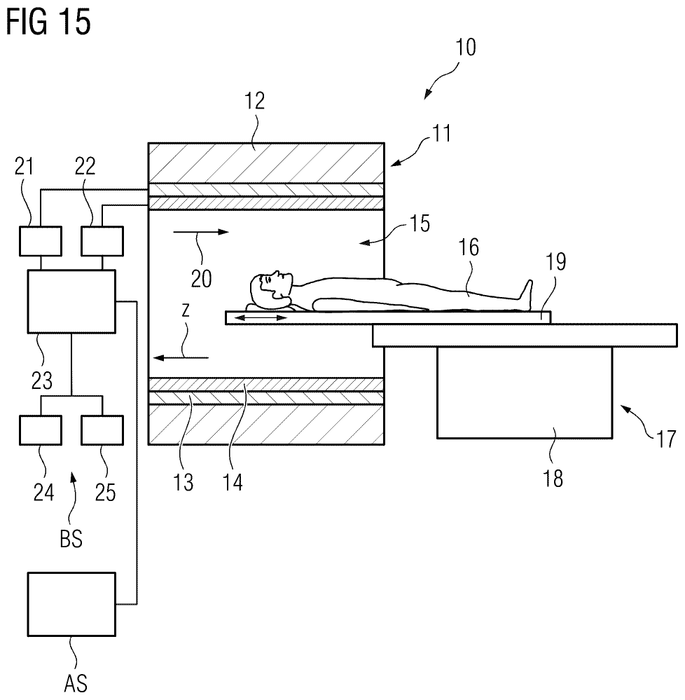

FIG. 15 is a schematic representation of a magnetic resonance device 10. The magnetic resonance device 10 comprises a magnet unit 11 with a base magnet 12, a gradient coil unit 13 and a high-frequency antenna unit 14. Furthermore, the magnetic resonance device 10 has a patient receiving area 15 for receiving a patient 16 for a magnetic resonance examination. The patient receiving area 15 in the present exemplary embodiment has a cylindrical design and is surrounded cylindrically in a circumferential direction by the magnet unit 11. Essentially, however, a deviating design of the patient receiving area 15 is conceivable at any time.

For positioning of the patient 16, e.g. an area of the patient 16 to be examined, within the patient receiving area 15, the magnetic resonance device 11 has a patient support device 17. The patient support device 17 has a base unit 18 and a patient table 19 that is movable in relation to the base unit 18. The patient table 19 is designed for positioning the patient 16, e.g. the area of the patient 16 to be examined, movably within the patient receiving area 15. In an embodiment, the patient table 19 is movably supported in the direction of a longitudinal extension of the patient receiving area 15 and/or in the z-direction.

The base magnet 12 of the magnet unit 11 is designed for generating a strong and constant base magnet field 20. At the same time, the base magnet 12 may for example be designed as a superconducting base magnet 12 or also as a permanent magnet. The gradient coil unit 13 of the magnet unit 11 is designed for generating magnetic field gradients, used for spatial encoding during imaging. The gradient coil unit 13 is controlled by means of a gradient control unit 21 of the magnetic resonance device 10. The RF antenna unit 14 of the magnet unit 11 is designed to stimulate a polarization, that occurs in the base magnet field 20 generated by the base magnet 12. The RF antenna unit 14 is controlled by a high-frequency antenna control unit 22 of the magnetic resonance device 10 and irradiates high-frequency magnetic resonance sequences into the patient receiving area 15 of the magnetic resonance device 10.

The gradient control unit 21 and the magnetic resonance device 10 have a system control unit 23 for controlling the base magnet 12 and for controlling the RF antenna unit 14 in each case. The system control unit 23 centrally controls the magnetic resonance device 10, such as executing a predetermined imaging gradient echo sequence. Furthermore, the system control unit 23 comprises an evaluation unit, not shown in more detail, for evaluation of medical image data, acquired during the magnetic resonance examination.

Moreover, the magnetic resonance device 10 comprises a user interface BS, linked to the system control unit 23. Control information such as imaging parameters, and reconstructed magnetic resonance images may be shown on a display unit 24, for example on at least one monitor, of the user interface BS for medical operating personnel. The user interface BS also has an input unit 25, by means of which information and/or parameters may be input by medical operating personnel during a measuring procedure.

For execution of the method for supporting and/or assisting a user by means of the assistance system AS when executing a measurement program MP during magnetic resonance data acquisition the magnetic resonance device comprises the assistance system AS. The assistance system AS is designed as described in FIG. 2 or FIG. 4. The assistance system AS is designed in the present exemplary embodiment separately from the system control unit 23, but is linked to this for data exchange. In an alternative design of the assistance system AS, this may also be arranged integrated in the system control unit 23.

The magnetic resonance device 10 described may of course comprise further components, which magnetic resonance devices 10 generally have. A general functioning of a magnetic resonance device 10 will also be known to the person skilled in the art, so that a detailed description of the further components may be dispensed with.

Although the disclosure has been illustrated and described in detail with the exemplary embodiments, the disclosure is not restricted by the examples disclosed and other variations may be derived therefrom by a person skilled in the art without departing from the protective scope of the disclosure.

The various components described herein may be referred to as “modules” or “units.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such units and/or modules, as applicable and relevant, may alternatively be referred to herein as “circuitry,” “controllers,” “processors,” or “processing circuitry,” or alternatively as noted herein.

What is claimed is:

1. A computer-implemented method for executing a measurement program during magnetic resonance data acquisition, comprising:
selecting a measurement program;
executing the selected measurement program to acquire magnetic resonance data and/or reconstruct image data from the acquired magnetic resonance data;
acquiring measurement information prior to or during execution of the selected measurement program;
determining an item of evaluation information as a function of one or more of the magnetic resonance data, the reconstructed image data, and the measurement information;
generating an item of assistance information as a function of the item of evaluation information via an analysis module; and
outputting the assistance information via a user interface.

2. The method as claimed in claim 1, wherein the assistance information comprises status information of the analysis module.

3. The method as claimed in claim 1, wherein the analysis module comprises one or more of a quality analysis module, a clinical analysis module, a technical analysis module, and a general analysis module.

4. The method as claimed in claim 1, wherein the item of evaluation information is determined via a rule-based algorithm and/or machine learning-based algorithm.

5. The method as claimed in claim 1, further comprising:
determining, via a recommendation module based on the item of evaluation information, a suggestion for a measurement step of the measurement program.

6. The method as claimed in claim 1, wherein the measurement program comprises multiple measurement steps and, for one of the multiple measurement steps, support via an assistance system is selectable for a user.

7. The method as claimed in claim 1, wherein the measurement program comprises multiple sequentially-executed measurement steps, and
wherein, for one of the multiple measurement steps for which support via an assistance system has been selected, a subsequent measurement step is only started upon completion of the one of the multiple measurement steps.

8. The method as claimed in claim 1, wherein the measurement program comprises multiple measurement steps and, for each of the multiple measurement steps for which support via an assistance system has been selected, the support is performed in a quasi-real time mode or a real-time mode.

9. The method as claimed in claim 1, wherein the method is performed via an assistance system, and
wherein an exchange of one or more of the magnetic resonance data, the image data, the measurement information, the item of evaluation information, the item of assistance information, and an item of extended assistance information of a recommendation module is performed via a transaction manager of an assistance system.

10. The method as claimed in claim 1, wherein the method is performed via an assistance system comprising a configuration user interface to configure a selection of an analysis module or a recommendation module for a measurement step of the measurement program.

11. The method as claimed in claim 1, wherein the method is performed via an assistance system, and
wherein an analysis module or a recommendation module of the assistance system generates output data comprising a defined output data format.

12. A magnetic resonance device, comprising:
a patient receiving area configured to receive a patient for a magnetic resonance examination during which magnetic resonance data acquisition is performed; and
an assistance system configured to execute a measurement program during the magnetic resonance data acquisition by:
selecting a measurement program;
executing the selected measurement program to acquire magnetic resonance data and/or reconstruct image data from the acquired magnetic resonance data;
acquiring measurement information prior to or during execution of the selected measurement program;
determining an item of evaluation information as a function of one or more of the magnetic resonance data, the reconstructed image data, and the measurement information;
generating an item of assistance information as a function of the item of evaluation information; and
outputting the assistance information via a user interface.

13. A non-transitory computer readable medium having instructions stored thereon that, when executed via an assistance system of a magnetic resonance device, cause the magnetic resonance device to execute a measurement program during magnetic resonance data acquisition by:
selecting a measurement program;
executing the selected measurement program to acquire magnetic resonance data and/or reconstruct image data from the acquired magnetic resonance data;
acquiring measurement information prior to or during execution of the selected measurement program;
determining an item of evaluation information as a function of one or more of the magnetic resonance data, the reconstructed image data, and the measurement information;
generating an item of assistance information as a function of the item of evaluation information; and
outputting the assistance information via a user interface.
